(19) United States
(12) Patent Application Publication
(10) Pub. No.: US 2014/0031289 A1
(43) Pub. Date: Jan. 30, 2014

(54) POLY(ORGANOPHOSPHAZENE) CONTAINING DEGRADATION CONTROLLABLE IONIC GROUP, PREPARATION METHOD THEREOF AND USE THEREOF

(71) Applicants: Soo-Chang SONG, Seoul (KR); Young-Min Kim, Seoul (KR)

(72) Inventors: Soo-Chang SONG, Seoul (KR); Young-Min Kim, Seoul (KR)

(73) Assignee: Korea Institute of Science and Technology, Seoul (KR)

(21) Appl. No.: 13/569,737

(22) Filed: Aug. 8, 2012

(30) Foreign Application Priority Data
Jul. 30, 2012 (KR) 10-2012-0083104

(51) Int. Cl. C08G 79/02; A61K 47/34

(52) U.S. Cl. CPC C08G 79/025; A61K 47/34
USPC 514/11.4; 528/321; 525/434; 514/772.3; 514/772.1

(57) ABSTRACT

The present invention relates to a thermosensitive phosphazene-based polymer having a degradation controllable ionic group, a use thereof, and a use thereof as a material for delivering bioactive substances. The phosphazene-based polymer according to the present invention has the thermosensitivity of showing the temperature-dependent sol-gel phase transition. Thus, it forms a gel phase at the body temperature when it is injected into the body to make it easy to control the release of bioactive substances such as drugs, and has the functional groups capable of making chemical bonds such as ionic bond, covalent bond, coordinate bond, etc. with drugs and thus is excellent in bearing the drugs. Since it can control the degradation rate depending on the kind of ionic group, it can selectively control the release time depending on the characteristics of drugs. Furthermore, it has an excellent biocompatibility and thus is very useful as a material for delivery of bioactive substances such as drugs, etc.

### CROSS-REFERENCE TO RELATED APPLICATION

This application claims priority to and the benefit of Korean Patent Application No. 10-2012-0083104, filed with Korean Intellectual Property Office on Jul. 30, 2012, the entire contents of which are incorporated herein by reference.

### BACKGROUND OF THE INVENTION

(a) Field of the Invention

The present invention relates to a polyphosphazene-based hydrogel for drug delivery which contains a degradation controllable ionic group, a preparation method thereof and a use thereof.

(b) Description of the Related Art

An aqueous polymer solution of a thermosensitive polymer hydrogel maintains a sol-phase at a low temperature but changes to a gel-phase as temperature increases. Such sol-gel phase transition may also be observed reversibly. The thermosensitive polymer hydrogels are evaluated as being very promising as a material for delivery of injectable drugs since they have such advantages that the aqueous polymer solution thereof may be easily mixed with therapeutic agents, they form a gel phase having a three-dimensional structure at body temperature when they are conveniently injected into a desired region without any surgical operation, and they are capable of sustained release of drugs (Nature, 388, 860 (1997), U.S. Pat. No. 6,201,072).

However, when the above thermosensitive polymer hydrogel is used as a material for injectable drug delivery, drugs with a low molecular weight or high hydrophilicity are easily released through the three-dimensional network structure of the gel after being injected into the body together with the polymer. Thus, there are such problems that 30% or more of the hydrophilic drugs contained are released at an early stage, and the drug release is completed within a short time due to the high diffusion rate of the hydrophilic drugs in the body (Adv Drug Deliv Rev, 31, 197 (1998)).

In order to complement such problems, some functional groups have been indirectly introduced to make drugs be kept in the gel, or some thermosensitive polymer hydrogels having functional groups capable of making a direct chemical bond have been reported. However, basically, if the hydrogel is degraded in the body, it is rapidly released even though it is combined with a drug. Since the maintenance period of drug effect is different depending on the kind of drug, it is also important to control the degradation rate of hydrogel to comply with the drug selected.

The present inventors have already reported that the phosphazene-based polymers which are obtained by substituting the linear dichlorophosphazene polymers with amino acid ester and methoxy polyethylene glycol show the characteristics of thermosensitive polymers, i.e., show the state of an aqueous solution at a specific temperature or lower and the phase transition from the sol-phase to the gel-phase of a three-dimensional structure at a specific temperature or higher. Furthermore, these thermosensitive phosphazene-based polymers are gradually hydrolyzed in an aqueous solution (Macromolecules 32, 2188 (1999), Macromolecules 32, 7820 (1999), Macromolecules 35, 3876 (2002), Korean Patent Nos. 259,367 and 315,630, U.S. Pat. No. 6,319,984).

Although the phosphazene-based polymers disclosed in the above articles or patents have a functional group for drug delivery, the degradation rate can be controlled only when a substituent is combined to the polymer main chain during the main synthetic reaction. However, the degradation rate should be different depending on the characteristics of drugs. Thus, in order to avoid the inconvenience of considering the degradation rate from the first stage of drug design process, it is required to develop a biodegradable phosphazene-based polymer which can selectively control the degradation rate depending on the kind of ionic group substituted, even after the main synthetic reaction, and can bear a bioactive substance in the polymer hydrogel with showing the temperature-dependent sol-gel phase transition.

### SUMMARY OF THE INVENTION

An embodiment provides a polyphosphazene-based hydrogel for drug delivery which contains a degradation controllable ionic group, a preparation method thereof and a use thereof.

Another embodiment provides a hydrogel comprising a specific concentration of the biodegradable and thermosensitive phosphazene-based polymer having a degradation controllable ionic group, and showing the temperature-dependent sol-gel phase transition.

Still another embodiment provides a drug delivery composition comprising one or more biodegradable and thermosensitive phosphazene-based polymers having a degradation controllable ionic group.

Still another embodiment provides a drug delivery system comprising one or more biodegradable and thermosensitive phosphazene-based polymers having a degradation controllable ionic group and one or more selected from the group consisting of drugs and therapeutic cells.

Still another embodiment provides a drug delivery system comprising one or more biodegradable and thermosensitive phosphazene-based polymers having a degradation controllable ionic group, one or more selected from the group consisting of drugs and therapeutic cells and one or more additives.

### DETAILED DESCRIPTION OF THE EMBODIMENT

The present invention relates to a polyphosphazene-based polymer for drug delivery which contains a degradation controllable ionic group, a hydrogel comprising same, a preparation method thereof and a use thereof as a material for delivering bioactive substances.

The phosphazene-based polymer according to the present invention is biodegradable and has the thermosensitivity of showing the temperature-dependent sol-gel phase transition. Thus, when it is injected into the body, it forms a gel phase at the body temperature to make it easy to control the release of bioactive substances such as drugs. It also has the functional groups capable of making chemical bonds such as ionic bond, covalent bond, coordinate bond, etc. with drugs and thus is excellent in bearing the drugs, etc. Furthermore, since it can control the degradation rate depending on the kind of ionic groups, it can selectively control the release time depending on the characteristics of drugs. It is also excellent in biocompatibility and thus is very useful as a material for delivery of bioactive substances such as drugs, etc.

Hereinafter, the present invention will be explained more in detail.

In one aspect, the present invention provides a phosphazene-based polymer having the following Formula (1):

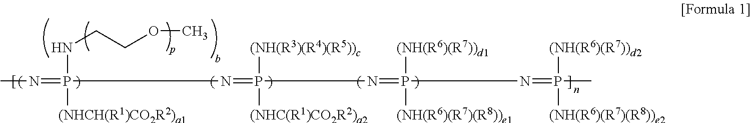

in which

p, which is the number of repeating units of ethylene glycol, ranges from 16 to 50,

NHCH(R′)CO₂R² is a hydrophobic amino acid ester, wherein R¹ is selected from the group consisting of H, CH₃, CH₂SH, CH(CH₃)₂, CH₂CH(CH₃)₂, CH(CH₃)C₂H₅, CH₂CH₂SCH₃, CH₂C₆H₅, CH₂C₆H₄OH and CH₂C₂NH₂C₆H₄, and R² is selected from the group consisting of CH₃, C₂H₅, C₃H₇, C₄H₉, CH₂C₆H₅ and CH₂CHCH₂,

NH(R³)(R⁴)(R⁵) is an amino acid, peptide, or depsipeptide ester, wherein R³ is CH(W), R⁴ is selected from the group consisting of CO₂, CO₂CH₂CO₂, CO₂CH(CH₃)CO₂ and CONHCH(X)CO₂, R⁵ is selected from the group consisting of H, CH₃ and C₂H₅, and W and X are independently selected from the group consisting of H, HCH₂, CH₃, CH(CH₃)₂, CH₂CH(CH₃)₂, CH(CH₃)C₂H₅, CH₂CH₂SCH₃, CH₂C₆H₅, CH₂C₂NH₂C₆H₄, CO₂C₂H₅, (CH₂)₂CO₂C₂H₅, CH₂OH, CH(CH₃)OH, CH₂C₆H₄OH, CH₂COOH, CH₂CH₂COOH, CH₂CONH₂, C₄H₈NH₂, C₃H₆NHC(═NH)NH₂, CH₂C₃N₂H₃ and CH₂SH,

NH(R⁶)(R⁷) and NH(R⁶)(R⁷)(R⁸) are substituents having a degradation controllable ionic group, wherein R⁶ is a divalent functional group of a hydroxy-containing compound, R⁷ is a monovalent or divalent functional group of a compound selected from the group consisting of dicarboxylic acid-based compounds having 3 to 30 carbon atoms, specifically 3 to 9 carbon atoms, more specifically 3 to 6 carbon atoms, and R⁸ is selected from the group consisting of a protecting group and NH₂CH(SH)CO₂H, NH₂(CH₂)_{q}SH, NH₂(CH₂CH₂NH)ᵣH, [NH₂CH(C₄H₈NH₂)CO]ᵣOH, [NH₂CH[(CH₂)₃C(═NH)(NH₂)]CO]ᵣOH, [OCH₂CH₂CH₂CH₂CH₂N(CH₂CH₂CO₂CH₂CH₂)₂]ᵣ, folic acid, hyaluronic acid, cyclodextrin, imidazole-based compound, anticancer agent, histidine, lysine, arginine, cysteine, thiolalkylamine (e.g., having 1 to 50 carbon atoms), spermine, spermidine, or polyethyleneimine, polyhistidine, polylysine or polyarginine having various weight average molecular weights, or protamine, heparin, chitosan, and peptide (comprising 1 to 20 amino acids, e.g., RGD or RGD derivative which is a peptide consisting of 4 to 5 amino acids comprising RGD, e.g., RGDS, RGDY, GRGDS, GRGDY, etc.),

q, which is the number of repeating units of methylene, ranges from 1 to 20,

r, which is the number of repeating units of ethyleneimine, lysine, aminoester, or arginine, ranges from 1 to 18000, wherein said ethyleneimine, lysine, aminoester or arginine is not limited in the molecular weight, but the weight average molecular weight thereof may be, for example, ranging from 50 to 100,000,

a₁, a₂, b, c, d₁, d₂, e₁ and e₂ represent the content of each substituent, wherein a₁, a₂, b, d₁ and d₂ respectively range from 0.01 to 1.9, c, e₁ and e₂ respectively range from 0 to 1.9, and a₁+a₂+b+c+d+d₂+e₁+e₂=2.0,

n, which is the degree of polymerization of the polyphosphazene, ranges from 3 to 100000.

In the above Formula (1), folic acid, hyaluronic acid, polyhistidine, cyclodextrin, heparin, chitosan and protamine which can be used as R⁸ are not limited in the molecular weight, but the weight average molecular weight thereof may be, for example, ranging from 50 to 100,000.

More preferably, the divalent functional group of a hydroxy-containing compound of R⁶ may be a divalent functional group derived from the hydroxy-containing compound. R⁶ may be a compound froduced by removing one hydrogen from the alkyl group or NH group from the amino acid and removing one hydrogen from the hydroxy group, in the hydroxyalkyl or hydroxy-containing amino acid, thereby acting as a divalent function group. For example, it may be selected from the group consisting of a divalent functional group derived from an alcohol (e.g., aminoethanol, aminopropanol, aminobutanol, aminopentanol, etc.) having straight-chain or branched alkyl which has 1 to 30 carbon atoms, preferably 1 to 10 carbon atoms, and is unsubstituted or substituted by one or more substituents selected from the group consisting of halogen, C₁-C₁₂-alkoxy, acryloyloxy and amino acid (i.e., one hydrogen from alkyl group and one hydrogen from hydroxy group are removed); a divalent functional group derived from an amino acid having hydroxy group (e.g., tyrosine, serine, threonine) (i.e., NH from amino acid and one hydrogen from hydroxy group are removed); etc.

The monovalent functional group of the dicarboxylic acid-based compound is used as R⁷ of NH(R⁶)(R⁷) and is in the form that hydroxy is removed from one of the two carboxyl groups acid-based compound is used as R⁷ of NH(R⁶)(R⁷)(R⁸) and is the form that each of the hydroxy groups (a total of two) is removed from each of the two carboxyl groups of the dicarboxylic acid-based compound. The dicarboxylic acid-based compounds are those inducing ionic groups that can control the degradation rate of polymers and may have a linear structure of all the conventionally used cyclic anhydrides, for example, a linear structure of cyclic anhydrides having 3 to 30 carbon atoms, specifically 3 to 9 carbon atoms, more specifically 3 to 6 carbon atoms. Unlimited examples thereof may be one or more selected from the group consisting of methylsuccinic acid, 3-3-dimethylglutaric acid, phenylsuccinic acid, aconitic acid, dimethylmaleic acid, itaconic acid, diglycolic acid, citraconic acid, glutaric acid, succinic acid, maleic acid, 2,2-dimethylsuccinic acid, 3-methylglutaric acid, phenylmaleic acid, 2-phenylglutaric acid, dodecenylsuccinic acid, dimethylmaleic acid, N—Z-L-aspartic acid, thiodiglycolic acid, tetrafluorosuccinic acid, cis-aconitic acid, 1-cyclopenten-1,2-dicarboxylic acid, phthalic acid, 3,6-dichlorophthalic acid, adipic acid, etc. Preferably, the dicarboxylic acid-based compounds may have 3 to 9 carbon atoms and may be one or more selected from the group consisting of succinic acid, maleic acid, glutaric acid, adipic acid and methylsuccinic acid. Most preferably, they are dicarboxylic acid-based compounds having 3 to 6 carbon atoms, for example, one or more selected from the group consisting of succinic acid, glutaric acid and adipic acid. As the dicarboxylic acid-based compounds have the more carbon numbers, they have the higher hydrophobicity, causing the harder gel degradation. On the contrary, as the dicarboxylic acid-based compounds have the less carbon numbers, they have the lower hydrophobicity, causing too rapid degradation. Thus, it is desirable that the dihydroxy dicarboxylic acid-based compounds have the carbon number in the above stated range in order to adequately control the degradation rate of the polymer.

The imidazole-based compound that can be used as R⁸ may be all the compounds having imidazole group, and for example, may be one or more selected from the group consisting of dacarbazine, 1-(3-aminopropyl)imidazole, methylhistamine dihydrochloride, 4-(1H-imidazol-1-yl)aniline, histamine, imiquimod, biotin ethylenediamine, 2-(2-methylimidazolyl)ethylamine dihydrochloride, 5-amino-4-imidazolecarboxamide hydrochloride, 5-aminoimidazole-4-carboxamide, 4-imidazoleacrylic acid, 4-imidazolecarboxylic acid, 2-iminobiotin, L-(+)-ergothioneine, 4,5-imidazoledicarboxylic acid, 1-(2-hydroxyethyl)imidazole, 4(5)-(hydroxymethyl)imidazole, 4-imidazolemethanol hydrochloride, etanidazole, 4-(imidazol-1-yl)phenol, HMMNI (2-hydroxymethyl-1-methyl-5-nitro-1H-imidazole), 2-mercaptoimidazole, 1-(4-hydroxybenzyl)imidazole-2-thiol, thiabendazole, 1,1′-thiocarbonyldiimidazole, 2-mercapto-1-methylimidazole, methimazole, 1-(2,3,5,6-tetrafluorophenyl)imidazole, 1-(heptafluorobutyryl)imidazole, 1-(pentafluoropropionyl)imidazole, 1-(trifluoroacetyl)imidazole, 1-(trifluoromethanesulfonyl)imidazole, 1-[2-(trifluoromethyl)phenyl]imidazole, 2-bromo-1H-imidazole, 2-butyl-4-chloro-5-(hydroxymethyl)imidazole, 2-butyl-5-chloro-1H-imidazole-4-carboxaldehyde, 2-chloro-1H-imidazole, 4-(4-bromophenyl)-1H-imidazole, 4-(4-chlorophenyl)-1H-imidazole, 4-(4-fluorophenyl)-1H-imidazole, 5-bromo-1-methyl-1H-imidazole, 6-bromo-1H-benzimidazole, cyazofamid, imazalil, ketoconazole, fenobam, imazalil sulfate, losartan potassium, neurodazine, nutlin-3, SB 220025 trihydrochloride, SB 202190 (4-(4-fluorophenyl)-2-(4-hydroxyphenyl)-5-(4-pyridyl)-1H-imidazole), PD 169316 (4-(4-fluorophenyl)-2-(4-nitrophenyl)-5-(4-pyridyl)-1H-imidazole), SB 239063 (trans-1-(4-hydroxycyclohexyl)-4-(4-fluorophenyl)-5-(2-methoxypyridimidin-4-yl)imidazole), tioconazole, triflumizole, 2,4,5-tribromoimidazole, 5-chloro-1-methyl-4-nitroimidazole, 2-ethyl-4-methyl-1H-imidazole-1-propanenitrile, 4,5-dicyanoimidazole, 5-ethynyl-1-methyl-1H-imidazole, etc.

The anticancer agent that can be used as R⁸ may be one or more selected from the group consisting of paclitaxel, doxorubicin, 5-fluorouracil, cisplatin, carboplatin, oxaliplatin, tegafur, irinotecan, docetaxel, cyclophosphamide, cemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansine, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassuim, medroxypogesterone acetate, epirubicin, letrozole, pirarubicin, topotecan, altretamine, toremifene citrate, BCNU, taxotere, actinomycin D, anasterozole, belotecan, imatinib, floxuridine, gemcitabine, hydroxyurea, zoledronate, vincristine, flutamide, valrubicin, streptozocin, polyethylene glycol-conjugated anticancer agents wherein polyethylene glycol is conjugated to the above anticancer agents, synthetic analogs thereof, and the above anticancer agents which are further modified or those having the same therapeutic effect.

Among all the compounds defined herein as a substituent, those described in the form of a substitutable functional group themselves are included in the structure of Formula (1). If the compound is described in the form of a complete compound, not a functional group, it can be included in the structure of Formula (1) in the form that one or two H and/or OH groups (if appropriate, NH group) are removed in order to be combined as a substituent. The form that one or two H and/or OH groups (if appropriate, NH group) are removed from the compound in order to be combined as a substituent can be easily understood by a person skilled in the art to which the present invention pertains from the structure of Formula (1).

For better understanding of the structure of Formula (1), substituents of the phosphazene-based polymers having the functional groups according to the present invention are summarized in the following Table 1.

TABLE 1

| Substituent | Kinds of Substituents | |
|---|---|---|
| NHCH(R¹)CO₂R² | R¹ | H, HCH₂, CH₃, CH₂SH, CH₂CH(CH₃)₂, CH₂CH(CH₃)₂, CH(CH₃)C₂H₅, |
| | | CH₂CH₂SCH₃, CH₂C₆H₅, CH₂C₆H₄OH, or CH₂C₂NH₂C₆H₄ |
| | R² | CH₃, C₂H₅, C₃H₇, C₄H₉, CH₂C₆H₅ or CH₂CHCH₂ |
| | Example | Phenylalanine ethyl ester in the case that R¹ = CH₂C₆H₅ and R² = C₂H₅,; or |
| | | glycine benzyl ester in the case that R¹ = H and R² = CH₂C₆H₅ |
| NH(R³)(R⁴)(R⁵) | R³ | CH(W) |
| | R⁴ | CO₂, CO₂CH₂CO₂, CO₂CH(CH₃)CO₂ or CONHCH(X)CO₂ |
| | R⁵ | H, CH₃ or C₂H₅ |
| | W, X | H, CH₃, CH(CH₃)₂, CH₂CH(CH₃)₂, CH(CH₃)C₂H₅, CH₂CH₂SCH₃, CH₂C₆H₅, |
| | | CH₂C₂NH₂C₆H₄, CO₂C₂H₅, (CH₂)₂CO₂C₂H₅, CH₂OH, CH(CH₃)OH, |
| | | CH₂C₆H₄OH, CH₂COOH, CH₂CH₂COOH, CH₂CONH₂, C₄H₈NH₂, |
| | | C₃H₆NHC(═NH)NH₂, CH₂C₃N₂H₃ or CH₂SH |
| | Example | Ethyl-2-(O-glycyl)glycolate in the case that R³ = CH₂, R⁴ = CO₂CH₂CO₂ and |
| | | R⁵ = C₂H₅; ethyl-2-(O-glycyl)lactate in the case that R³ = CH₂CO₂, |
| | | R⁴ = CO₂CH(CH₃)CO₂ and R⁵ = C₂H₅; glycine in the case that R³ = CH₂, R⁴ = CO₂ |
| | | and R⁵ = H; or glycylglycine in the case that R³ = CH₂, R⁴ = CO₂NHCH₂CO₂ and |
| | | R⁵ = H |
| NH(R⁶)(R⁷) | R⁶ | Amino acid having hydroxyalkyl group or hydroxy group |
| and | | (aminoalcohol-based compound) |
| NH(R⁶)(R⁷)(R⁸) | R⁷ | Dicarboxylic acid-based compound |
| | R⁸ | Protecting group and NH₂CH(SH)CO₂H, NH₂(CH₂)_{q}SH, NH₂(CH₂CH₂NH)ᵣH, |
| | | [NH₂CH(C₄H₈NH₂)CO]ᵣOH, [NH₂CH[(CH₂)₃C(═NH)(NH₂)]CO]ᵣOH, |
| | | [OCH₂CH₂CH₂CH₂CH₂N(CH₂CH₂CO₂CH₂CH₂)₂]ᵣ, folic acid, hyaluronic acid, |
| | | cyclodextrin, imidazole-based compound, anticancer agent, histidine, lysine, |
| | | arginine, cysteine, RGD, RGD derivative, thiolalkylamine (e.g., having 1 to 50 |
| | | carbon atoms), spermine, spermidine, o polyethyleneimine, polyhistidine, |
| | | polylysine, polyarginine or protamine having a variety of weight average |
| | | molecular weight, heparin, chitosan or protamine |
| | Ex. | NHCHCH₂CH₂OCOCH₂COOH in the case that R⁶ = divalent functional group |
| | | of propanol and R⁷ = maleic acid; |
| | | NHCHCH₂CH₂CH₂CH₂OCOCH₂CH₂CH₂COOH in the case that R⁶ = divalent |
| | | functional group of pentanol and R⁷ = glutaric acid; |
| | | NHCHCH₂OCOCH₂CH₂CH₂CH₂CONH-heparin in the case that R⁶ = divalent |
| | | functional group of ethanol, R⁷ = adipic acid and R⁸ = heparin |

In the above phosphazene-based polymer, hydrophobic amino acid ester and hydrophilic methoxy polyethylene glycol having the molecular weight of 750 to 2500 may be introduced to the linear dichlorophosphazene polymer for the purpose of endowing the polymer with the thermosensitivity and biodegradability, and some amino acid, peptide or depsipeptide ester which can control the degradation rate of the polymer may be partially introduced. Also, a functional group may be introduced to the phosphazene-based polymer of the present invention in such a manner that a substituent having the functional group such as hydroxy, amide, amino, thiol or carboxyl in its side chain is directly introduced to the polymer main chain, or the amino acid ester or peptide ester whose functional group is protected is introduced to the polymer main chain and then the protecting group is removed.

The protecting group that can be used for R⁸ in Formula (1) may be all the protecting groups conventionally used for protecting each functional group, and unlimited examples thereof are those described in the following Tables 2a to 2e.

TABLE 2

| Functional | |
|---|---|
| group | Protecting group (R′ = R⁸) |
| Carboxyl | Fluorenylmethyl ester, Methoxymethyl ester (CH₂OCH₃), Methylthiomethyl ester (CH₂SCH₃), |
| group | Tetrahydrofuranyl ester, Methoxyethoxymethyl ester (CH₂OCH₂CH₂OCH₃), 2- |
| (RCOOR′) | (trimethylsilyl)ethoxymethyl ester (CH₂OCH₂CH₂Si(CH₃)₃), Benzyloxymethyl ester (CH₂OCH₂C₆H₅), |
| | Pivaloxyloxymethyl ester (CH₂O₂CC(CH₃)₃), Phenylacetoxymethyl ester (CH₂O₂CCH₂Ph), |
| | Triisopropylsilylmethyl ester (CH₂Si-i-Pr₃), Cyanomethyl ester (CH₂CN), Acetol ester (CH₂COCH₃), |
| | Phenacyl ester (CH₂COC₆H₅), p-Bromophenacyl ester (CH₂COC₆H₄-p-Br), α-Methylphenacyl ester |
| | (CH(CH₃)COC₆H₅). p-Methoxyphenacyl ester (CH₂COC₆H₄-p-OCH₃), Desyl ester, Carboxamidomethyl |
| | ester (CH₂CONH₂), p-Azobenzenecarboxamidomethyl ester (CH₂(O)CNHC₆H₄N═NC₆H₅), N- |
| | Phthalimidomethyl ester, 2,2,2-Trichloroethyl ester (CH₂CCl₃), 2-Haloethyl ester (CH₂CH₂X, X = I, Br, |
| | Cl), ω-Chloroalkyl ester ((CH₂)ₙCl, n = 4, 5), 2-(trimethylsilyl)ethyl ester (CH₂CH₂Si(CH₃)₃), 2- |
| | Methylthioethyl ester (CH₂CH₂SCH₃), 1,3-Dithianyl-2-methyl ester, 2-(p-Nitrophenylsulfenyl)ethyl |
| | ester (CH₂CH₂SC₆H₄-p-NO₂), 2-(p-Toluenesulfonyl)ethyl ester (CH₂CH₂SO₂C₆H₄-p-CH₃), 2-(2′- |
| | Pyridyl)ethyl ester (CH₂CH₂-2-C₅H₄N), 2-(p-Methoxyphenyl)ethyl ester (CH₂CH₂C₆H₄O-p-CH₃), 2- |
| | (diphenylphosphino)ethyl ester (CH₂CH₂P(C₆H₅)₂), 1-Methyl-1-phenylethyl ester (C(CH₃)₂C₆H₅), 2-(4- |
| | Acetyl-2-nitrophenyl)ethyl ester, 2-Cyanoethyl ester (CH₂CH₂CHN), t-Butyl ester (C(CH₃)₃), 3-Methyl- |
| | 3-pentyl ester (CCH₃(C₂H₄)₂), Dicyclopropylmethyl ester, 2,4-Dimethyl-3-pentyl ester (CH(i-Pr)₂), |
| | Cyclopentyl ester (c-C₅H₉), Cyclohexyl ester (c-C₆H₁₁), Allyl ester (CH₂CH═CH₂), Methallyl ester |
| | (CH₂(CH₃)C═CH₂), 2-Methylbut-3-en-2-yl ester (C(CH₃)₂CH═CH₂), 3-Methylbut-2-enyl ester |
| | (CH₂CH═C(CH₃)₂), 3-Buten-1-yl ester (CH₂CH₂CH═CH₂), 4-(Trimethylsilyl)-2-buten-1-yl ester |
| | (CH₂CH═CHCH₂Si(CH₃)₃), Cinnamyl ester (CH₂CH═CHC₆H₅), α-Methylcinnamyl ester |
| | (CH(CH₃)CH═CHC₆H₅), Prop-2-ynyl ester (CH₂C≡CH), Phenyl ester (C₆H₅), 2,6-Dimethylphenyl ester, |
| | 2,6-Diisopropylphenyl ester, 2,6-Di-t-butyl-4-methylphenyl ester, 2,6-Di-t-Butyl-4-methoxyphenyl |
| | ester, p-(Methylthio)phenyl ester (C₆H₄-p-SCH₃), Pentafluorophenyl ester (C₆F₅), Benzyl ester |
| | (CH₂C₆H₅), Triphenylmethyl ester (C(C₆H₅)₃), Diphenylmethyl ester (CH(C₆H₅)₂) Bis(o- |
| | nitrophenyl)methyl ester (CH(C₆H₄-o-NO₂)₂), 9-Anthrylmethyl ester (CH₂-9-Anthryl), 2-(9,10- |
| | Dioxo)anthrylmethyl) ester, 5-dibenzosuberyl ester, 1-Pyrenylmethyl ester, 2-(trifluoroaceticmthyl)-6- |
| | chromonylmethyl ester, 2,4,6-Trimethylbenzyl ester (CH₂C₆H₂-2,4,6-(CH₃)₃), p-Bromobenzyl ester |
| | (CH₂C₆H₄-p-Br), o-Nitrobenzyl ester (CH₂C₆H₄-o-NO₂), p-Nitrobenzyl ester (CH₂C₆H₄-p-NO₂), p- |
| | Methoxybenzyl ester (CH₂C₆H₄-p-OCH₃), 2,6-Dimethoxybenzyl ester (CH₂C₆H₃-2,6-(OCH₃)₂, 4- |
| | (Methylsulfinyl)benzyl ester (CH₂C₆H₄(O)S-4-CH₃), 4-Sulfobenzyl ester (CH₂C₆H₄SO₃⁻Na⁺), 4- |
| | Azidomethoxybenzyl ester (CH₂C₆H₄OCH₂N₃), 4-{N-[1-(4,4-Dimethyl-2,6-dioxocyclohexylidene)-3- |
| | methlbutyl]amino}benzyl ester, Piperonyl ester, 4-Picolyl ester (CH₂-4-pyridyl), p-P-Benzayl ester |
| | (CH₂C₆H₄-p-P), Trimethylsilyl ester (Si(CH₃)₃), Triethylsilyl ester (Si(C₂H₅)₃), t-Butyldimethylsilyl |
| | ester (Si(CH₃)₂C(CH₃), i-Propyldimethylsilyl ester (Si(CH₃)₂CH(CH₃)₂), Phenyldimethylsilyl ester |
| | (Si(CH3)₂C₆H₅), Di-t-butylmethylsilyl ester (SiCH₃(t-Bu)₂), Triisopropylsilyl ester |
| Thiol | S-Alkyl thioether (CₙH₂ₙ₊₁), S-Benzyl thioether (CH₂Ph), S-p-Methoxylbenzyl thioether (CH₂C6H4-p- |
| group | OCH₃), S-o- or p-Hydroxy-or-Acetoxybenzyl thioether (CH₂C6H4-o-(or p-)-OR′, R′ = H or Ac), S-p- |
| (RSR′) | Nitrobenzyl thioether (CH₂C₆H₄-p-NO₂), S-2,4,6-Trimethylbenzyl thioether (CH₂C₆H₂-2,4,6-Me₃), S- |
| | 2,4,6-Trimethoxybenzyl thioether (CH₂C₆H₂-2,4,6-(OMe)₃), S-4-Picolyl thioether (CH₂-4-pyridyl), S-2- |
| | Quinolinylmethyl thioether, S-2-Picolyl N-Oxide thioether (CH₂-2-pyridyl N-Oxide), S-9- |
| | Anthrylmethyl thioether (CH₂-9-anthtyl), S-9-Fluorenylmethyl thioether, S-Xanthenyl thioether, S- |
| | Ferrocenylmethyl thioether, S-Diphenylmethyl thioether (CH(C₆H₅)₂), S-Bis(4-methoxyphenyl)methyl |
| | thioether (CH(C₆H₄-4-OCH₃)₂), S-Bis(4-methoxyphenyl)phenylmethyl thioether, S-5-Dibenzosuberyl |
| | thioether, S-Triphenylmethyl thioether (C(C₆H₅)₃), S-Diphenyl-4-pyridylmethyl thioether (C(C₆H₅)₂-4- |
| | Pyridyl), S-Phenyl thioether (C₆H₅), S-2,4-Dinitrophenyl thioether (C₆H₃-2,4-(NO₂)₂), S-t-Butyl |
| | thioether (C(CH₃)₃), S-1-Adamantyl thioether, S-Methoxymethyl monothioacetal (CH₂OCH₃), S- |
| | Isobutoxymethyl monothioacetal (CH₂OCH₂CH(CH₃)₂), S-Benzyloxymethyl monothioacetal (CH₂OBn), |
| | S-2-Tetrahhydropyranyl monothioacetal, S-Benzylthiomethyl dithioacetal (CH₂SCH₂C₆H₅), S- |
| | Phenylthiomethyl dithioacetal (CH₂SC₆H₅), S-Acetamidomethyl thioacetal (CH₂NHCOCH₃), S- |
| | Trimethylacetamidomethyl thioacetal (CH₂NHCOC(CH₃)₃), S-Benzamidomethyl |
| | (thioacetalCH₂NHCOC₆H₅), S-Allyloxycarbonylaminomethyl thioacetal (CH₂NH(O)COCH₂CH═CH₂), |
| | S-Phenylacetamidomethyl thioacetal (CH₂NH(O)CCH₂C₆H₅), S-Phthalimidomethyl thioacetal, S- |
| | Acetyl, S-Carboxy, and S-Cyanomethyl thioether (CH₂X, X = —COCH₃, —CO₂H, —CN), S-(2-Nitro-1- |
| | phenyl)ethyl thioether (CH(C₆H₅)CH₂NO₂), S-2-(2,4-Dinitrophenyl)ethyl thioether, S-2-(4′- |
| | Pyridyl)ethyl thioether (CH₂CH₂NC₄H₄), S-2-Cyanoethyl thioether (CH₂CH₂CN), S-2- |
| | (Trimethylsilyl)ethyl thioether (CH₂CH₂TMS), S-2,2-Bis(carboethoxy)ethyl thioether |
| | (CH₂CH(COOC₂H₅)₂), S-(1-m-Nitrophenyl-2-benzoyl)ethyl thioether (CH(C₆H₄-m-NO₂)CH₂COC₆H₅), |
| | S-2-phenylsulfonylethyl thioether (CH₂CH₂SO₂Ph), S-1-(4-Methylphenylsulfonyl)-2-methylprop-2-yl |
| | thioether (C(CH₃)₂CH₂SO₂C₆H₄-4-CH₃), Triisopropylsilyl thioether, S-Acetyl derivatives (COCH₃), S- |
| | Benzoyl derivatives (COC₆H₅), S-Trifluoroaceticacetyl derivatives(COCF₃), S-2,2,2- |
| | Trichloroethoxycarbonyl derivatives (COOCH₂CCl₃), S-t-Butoxycarbonyl derivatives (COOC(CH₃)₃), |
| | S-Benzyloxycarbonyl derivatives (COOCH₂C₆H₅), S-p-Methoxybenzyloxycarbonyl derivatives |
| | (COOCH₂C₆H₄-p-OCH₃), S-(N-Ethylcarbamate)(CONHC₂H₅), S-(N-Methoxymethylcarbamate) |
| | (CONHCH₂OCH₃), S-Ethyl disulfide (SC₂H₅), S-t-Butyl disulfide (SC(CH₃)₃) |
| Hydroxy | Methyl ether (CH₃), Methoxymethyl ether (CH₂OCH₃), Methylthiomethyl ether (CH₂SCH₃), |
| group | (Phenyldimethylsilyl)methoxymethyl ether (CH₂OCH₂Si(CH₃)₂C₆H₅), Benzyloxymethyl ether |
| (ROR′) | (CH₂OCH₂Ph), p-Methoxybenzyloxymethyl ether (CH₂OCH₂C₆H₄O-p-Me), p-Nitrobenzyloxymethyl |
| | ether (CH₂OCH₂C₆H₄-4-NO₂), o-Nitrobenzyloxymethyl ether (CH₂OCH₂C₆H₄-2-NO₂), (4- |
| | Methoxyphenoxy)methyl ether (CH₂OC₆H₄-4-OCH₃), Guaiacolmethyl ether (CH₂OC₆H₄-2-OMe), t- |
| | Butoxymethyl ether (CH₂O-t-Bu), 4-Pentenyloxymethyl ether (CH₂OCH₂CH₂CH₂CH═CH₂), |
| | Siloxymethyl ether (CH₂OSiR′R″, R′ = t-Bu, R″ = Me; R′ = Thexyl, R″ = Me; R′ = t-Bu, R″ = Ph), 2- |
| | Methoxyethoxymethyl ether (CH₂OCH₂CH₂OCH₃), 2,2,2-Trichloroethoxymethyl ether |
| | (CH₂OCH₂CCl₃), Bis(2-chloroethoxy)methyl ether (CH(OCH₂CH₂Cl)₂), 2-(Trimethylsilyl)ethoxymethyl |
| | ether (CH₂OCH₂CH₂SiMe₃), Methoxymethyl ether, Tetrahydropyranyl ether, 3-Bromotetrahydropyranyl |
| | ether, Tetrahydrothiopyranyl ether, 1-Methoxycyclohexyl ether, 4-Methoxytetrahydropyranyl ether, 4- |
| | Methoxytetrahydrothiopyranyl ether, 1-[(2-Chloro-4-methyl)phenyl]-4-methoxypiperidin-4-yl ether, 1- |
| | (2-Fluorophenyl)-4-methoxypiperidin-4-yl ether, 1,4-Dioxan-2-yl ether, Tetrahydrofuranyl ether, |
| | Tetrahydrothiofuranyl ether, 2,3,3a,4,5,6,7,7a-octahydro-7,8,8-trimethyl-4,7-methanobenzofuran-2-yl |
| | ether, 1-Ethoxyethyl ether (CH(OC₂H₅)CH₃), 1-(2-Chloroethoxy)ethyl ether (CH(CH₃)OCH₂CH₂Cl), 1- |
| | [2-(Trimethylsilyl)ethoxy]ethyl ether, 1-Methyl-1-methoxyethyl ether (C(OCH₃)(CH₃)₂), 1-Methyl-1- |
| | benzyloxyethyl ether (C(OBn)(CH₃)₂), 1-Methyl-1-benzyloxy-2-fluoroethyl ether (C(OBn)(CH₂F)(CH₃), |
| | 1-Methyl-1-phenoxyethyl ether (C(OPh)(CH₃)₂), 2,2,2-trichloroethyl ether (CH₂CCl₃), 1,1-Dianisyl- |
| | 2,2,2-trichloroethyl ether, 1,1,1,3,3,3-Hexafluoro-2-phenylisopropyl ether (C(CHF₃)₂Ph), 2- |
| | Trimethylsilylethyl ether (CH₂SiMe₃), 2-(Benzylthio)ethyl ether (CH₂CH₂SBn), 2-(Phenylselenyl)ethyl |
| | ether (CH₂CH₂SePh), t-Butyl ether, Allyl ether (CH₂CH═CH₂), Propargyl ether (CH₂C≡CH), p- |
| | Methoxyphenyl ether (C₆H₄O-p-Me), p-Nitrophenyl ether (C₆H₄-p-NO₂), 2,4-Dinitrophenyl ether (C₆H₃- |
| | 2,4-(NO₂)₂), 2,3,5,6-Tetrafluoro-4-(trifluoroaceticmethyl)phenyl ether (C₆F₄CF₃), Benzyl ether (CH₂Ph), |
| | p-Methoxybenzyl ether (CH₂C₆H₄-p-OMe), 3,4-Dimethoxybenzyl ether (CH₂C₆H₃-3,4-(OMe)₂), o- |
| | Nitrobenzyl ether (CH₂C₆H₄-o-NO₂), p-Nitrobenzyl ether (CH₂C₆H₄-p-NO₂), p-Halobenzyl ether |
| | (CH₂C₆H₄-p-X, X = Br, Cl), 2,6-Dichlorobenzyl ether (CH₂C₆H₃-2,6-Cl₂), p-Cyanobenzyl ether |
| | (CH₂C₆H₄-p-CN), p-Phenylbenzyl ether (CH₂C₆H₄-p-C₆H₅), 2,6-Difluorobenzyl ether (CH₂C₆H₃F₂), p- |
| | Acylaminobenzyl ether (CH₂C₆H₃-p-NHCOR′), p-Azidobenzyl ether (CH₂C₆H₄-4-N₃),4-Azido-3- |
| | chlorobenxyl ether (CH₂C₆H₃-3-Cl-4-N₃), 2-Trifluoroaceticmethylbenzyl ether (CH₂C₆H₄-2-CF₃), p- |
| | (Methylsulfinyl)benzyl ether (CH₂C₆H₄-p-(MeS(O)), 2- and 4-Picolyl ether(CH₂C₅H₄N), 3-Methyl-2- |
| | picolyl N-Oxido ether, 2-Quinolinylmethyl ether, 1-Pyrenylmethyl ether, Diphenylmethyl ether |
| | (CHPh₂), p,p′-Dinitrobenzhydryl ether (CH(C₆H₄-p-NO₂)₂), 5-Dibenzosuberyl ether, Triphenylmethyl |
| | ether, p-Methoxyphenyldiphenylmethyl ether (C(Ph)₂C₆H₄-p-OMe), Di(p-methoxyphenyl)phenylmethyl |
| | ether (CPh(p-MeOC₆H₄)₂), Tri(p-methoxyphenyl)methyl ether (C(p-MeOC₆H₄)₃), 4-(4′- |
| | Bromophenacyloxy)phenyldiphenylmethyl ether (C(Ph)₂C₆H₄-p-(OCH₂(O)CC₆H₄-p-Br), 4,4′,4″- |
| | Tris(4,5-dichlorophthalimidophenyl)methyl ether, 4,4′,4″-Tris(levulinoyloxyphenyl)methyl) ether, |
| | 4,4′4″-Tris(benzoyloxyphenyl)methyl) ether, 4,4′-Dimethoxy-3″-[N-(imidazolylmethyl)]trityl ether, 4,4′- |
| | Dimethoxy,3″-[N-(imidazolylethyl)carbamoyl)trityl ether, 1,1-Bis(4-methoxyphenyl)-1-pytenylmethyl |
| | ether, 4-(17-tetrabenzo[a,c,g,i]fluorenylmethyl)-4′,4″-dimethoxytrityl ether, 9-Anthryl ether, 9-(9- |
| | Phenyl)xanthenyl ether, Tritylone ether, 1,3-Benzodithiolan-2-yl ether, Benzisothiazolyl-S,S-dioxido |
| | ether, Trimethylsilyl (e.g., Si(CH₃)₃) ether, Triethylsilyl (SiEt₃) ether, Triisopropylsilyl (Si(i-Pr)₃) ether, |
| | Dimethylisopropylsilyl (SiMe₂-i-Pr) ether, Diethylisopropylsilyl (SiEt₂-i-Pr) ether, Dimethylthesilyl |
| | ether ((CH₃)₂Si(CH₃)₂CCH(CH₃)₂), t-Butyldimethylsilyl ether (SiMe₂-t-Bu),t-Butyldiphenylsilyl ether |
| | (SiPh₂-t-Bu), Tribenxylsily ether (Si(CH₂C₆H₅)₃), Tri-p-xylylsilyl ether (Si(CH₂C₆H₄-p-CH₃)₃), |
| | Triphenylsilyl ether (SiPh₃), Diphenylmethylsily ether (SiMePh₂), Di-t-butylmethylsilyl ether (SiMe(t- |
| | Bu)₂),Tris(trimethylsilyl)silyl ether ([Si[Si(CH₃)₃]₃), (2-Hydroxystyryl)dimethylsilyl ether, (2- |
| | Hydroxystyryl)diisopropylsilyl ether, t-Butylmethoxyphenylsilyl ether (SiPh(OCH₃)-t-Bu), t- |
| | Butoxydiphenylsilyl ether (Si(t-OBu)Ph₂), Formate ester (CHO), Benzoylformate ester (COCOPh), |
| | Acetate ester (COCH₃), Chloroacetate ester (COCH₂Cl), Dichloroacetate ester (COCHCl₂), |
| | Trichloroacetate ester (COCCl₃), Trifluoroaceticacetate ester (COCF₃), Methoxyacetate ester |
| | (COCH₂OMe), Triphenylmethoxyacetate ester (COCH₂OCPh₃), Phenoxyaetate ester (COCH₂OPh), p- |
| | chlorophenoxyacetate ester (COCH₂OC₆H₄—p-Cl), phenylacetate ester (COCH₂Ph), p-P-Phenylacetate |
| | ester (COCH₂C₆H₄-p-P), Diphenylacetate ester (COCHPh₂), Nicotinate ester, 3-Phenylpropionate ester |
| | (COCH₂CH₂Ph), 4-Pentenoate ester (COCH₂CH₂CH═CH₂), 4-Oxopentanoate ester |
| | (COCH₂CH₂COCH₃), 4,4-(Ethylenedithio)pentanoate ester, 5-[3-Bis(4- |
| | methoxyphenyl)hydroxymethylphenoxy]levulinic acid ester, Pivaloate (COC(CH₃)₃) ester, Crotonate |
| | ester (COCH═CHCH₃), 4-Methoxycrotonate ester (COCH═CHCH₂OCH₃), Benzoate ester (COPh), p- |
| | Phenylbenzoate ester (COC₆H₄-p-C₆H₅), 2,4,6-Trimethylbenzoate ester (COC₆H₂-2,4,6-Me₃), Alkyl |
| | methyl carbonate (CO₂CH₃), Methoxymethyl carbonate (CO₂CH₂OCH₃), alkyl 9-fluorenylmethyl |
| | carbonate, Alkyl ethyl carbonate (CO₂Et), Alkyl 2,2,2-Trichloroethyl carbonate (CO₂CH₂CCl₃), 1,1- |
| | Dimethyl-2,2,2-trichloroethyl carbonate (CO₂C(CH₃)₂CCl₃), Alkyl 2-(trimethylsilyl)ethyl carbonate |
| | (CO₂CH₂CH₂SiMe₃), Alkyl 2-(phenylsulfonyl)ethyl caronate (CO₂CH₂CH₂SO₂Ph), Alkyl isobutyl |
| | carbonate (CO₂CH₂CH(CH₃)₂), Alkyl vinyl carbonate (CO₂CH═CH₂), Alkyl allyl carbonate |
| | (CO₂CH₂CH═CH₂), Alkyl p-nitrophenyl carbonate (CO₂C₆H₄—p-NO₂), Alkyl benzyl carbonate (CO₂Bn), |
| | Alkyl p-methoxybenzyl carbonate (CO₂CH₂C₆H₄—p-OMe), Alkyl 3,4-dimethoxybenzyl carbonate |
| | (CO₂CH₂C₆H₃-3,4-(OMe)₂), Alkyl o-nitrobenzyl carbonate (CO₂CH₂C₆H₄—o-NO₂), Alkyl p-nitrobenzyl |
| | carbonate (CO₂CH₂C₆H₄—p-NO₂), 2-Dansylethyl carbonate, 2-(4-Nitrophenyl)ethyl carbonate |
| | (CO₂CH₂CH₂C₆H₄—4-NO₂), 2-(2,4-dinitrophenyl)ethyl carbonate (CO₂CH₂CH₂C₆H₃-2,4-(NO₂)₂), 2- |
| | Cyano-1-phenylethyl carbonate (CO₂(C₆H₅)CHCH₂CN), Alkyl S-Benzyl thiocarbonate (COSCH₂Ph), |
| | Alkyl 4-ethoxy-1-naphthyl carbonate, Alkyl methyl dithiocarbonate (SCSCH₃), 2-iodobenzoate ester |
| | (COC₆H₄-2-I), 4-Azidobutyrate ester (CO(CH₂)₃N₃), 4-Nitro-4-methylpentanoate ester, o- |
| | (dibromomethyl)benzoate ester (COC₆H₄—o-(CHBr₂)), 2-Formylbenzenesulfonate ester, Alkyl 2- |
| | (methylthiomethoxy)ethyl carbonate (CO₂CH₂CH₂OCH₂SCH₃), 4-(Methylthiomethoxy)butyrate ester |
| | (CO(CH₂)₃OCH₂SCH₃), 2-(Methylthiomethoxymethyl)benzoate ester (COC₆H₄-2-(CH₂OCH₂SCH₃)), 2- |
| | (Chloroacetoxymethyl)benzioate ester, 2-[(2-chloroacetoxy)ethyl]benzoate ester, 2-[2- |
| | (Benzyloxy)ethyl]benzoate ester, 2-[2-(4-Methoxybenzyloxy)ethyl]benzoate ester, 2,6-Dichloro-4- |
| | methylphenoxyacetate ester, 2,6-Dichloro-4-(1,1,3,3-tetramethylbutyl)phenoxyacetate ester, 2,4-Bis(1,1- |
| | dimethylpropyl)phenoxyacetate ester, Chlorodiphenylacetate ester, Isobutyrate ester, Monosuccinoate |
| | ester, (E)-2-Methyl-2-Butenoate ester, o-(Methoxycarbonyl)benzoate ester, p-P-Benzoate ester, α- |
| | Naphthoate ester, Nitrate ester, Alkyl N,N,N′,N′-tetramethylphosphorodiamidate, 2-Chlorobenzoate |
| | ester, 4-Bromobenzoate ester, 4-Nitrobenzoate ester, 3,5-Dimethoxybenzoin carbonate, A wild and |
| | woolly photolabile fluorescent ester, Alkyl N-phenylcarbamate, Borate ester, Dimethylphosphinothioyl |
| | ester ((S)P(CH₃)₂), Alkyl 2,4-dinitrophenylsulfenate (SC₆H₃-2,4-(NO₂)₂), Sulfate, Allylsulfonate |
| | (SOCH₂CH═CH₂), Methanesulfonate (SO₂Me), Benzylsulfonate (SO₂Bn), Tosylate (SO₂C₆H₄CH₃),2- |
| | [(4-Nitrophenyl)ethyl]sulfonate (SO₂CH₂CH₂C₆H₄-4-NO₂) |
| Amino | Formamide (CHO), Acetamide (Ac), Chloroacetamide (COCH₂Cl), Trichloroacetamide (COCCl₃), |
| group | Trifluoroaceticacetamide (COCF₃), Phenylacetamide (COCH₂C₆H₅), 3-Phenylpropanamide |
| (RNR′) | (COCH₂CH₂C₆H₅), Pent-4-enamide ((O)CH₂CH₂CH═CH₂), Picolinamide (CO-2-pyridyl), 3- |
| | Pyridylcarboxamide (CO-3-Pyridyl), N-Benzoylphenylalanyl derivatives |
| | (COCH(NHCOC₆H₅)CH₂C₆H₅), Benzamide (COC₆H₅), p-Phenybenzamide (COC₆H₄—p-C₆H₅) |
| Amide | N-Allylamide (CH₂CH═CH₂), N-t-Butylamide (t-Bu), N-Dicyclopropylmethylamide (CH(C₃H₅)₂), N- |
| group | Methoxymethylamide (CH₂OCH₃), N-Methylthiomethylamide (CH₂SCH₃), N-Benzyloxymethylamide |
| (CORNR′) | (CH₂OCH₂C₆H₅), N-2,2,2-Trichloroethoxymethylamide (CH₂OCH₂CCl₃), N-t- |
| | Butyldimethylsiloxymethylamide (CH₂OSi(CH3)₂—y-C₄H₉), N-Pivaloyloxymethylamide |
| | (CH₂CO₂C(CH₃)₃), N-Cyanomethylamide (CH₂CHN), N-Pyrrolidinomethylamide, N-Methoxyamide |
| | (OMe), N-Benzyloxyamide (OCH₂C₆H₅), N-Methylthioamide (SMe), N-Triphenylmethylthioamide |
| | (SCPh₃), N-t-Butyldiethylsilylamide (Si(CH₃)₂—t-C₄H₉), N-Triisopropylsilylamide (Si(i-Pr)₃), N-4- |
| | Methoxyphenylamide (C₆H₄-4-OCH₃), N-4-(Methoxymethoxy)phenylamide (C₆H₄(OCH₃)₂), N-2- |
| | Methoxy-1-naphthylamide (C₁₀H₆-₂-OCH₃), N-Benzylamide (CH₂C₆H₅), N-4-Methoxybenzylamide |
| | (CH₂C₆H₄-4-OCH₃), N-2,4-Dimethoxybenzylamide N-3,4-Dimethoxybenzylamide (CH₂C₆HH₃-2,4(3,4)- |
| | (OCH₃)₂), N-2-Acetoxy-4-methoxybenzylamide (CH₂C₆HH₃-4-OMe-2-Ac), N-o-nitrobenzylamide |
| | (CH₂C₆H₄-2-NO₂), N-Bis(4-methoxyphenyl)methylamide (CH(C₆H₄-4-OMe)₂), N-Bis(4- |
| | (methoxyphenyl)phenylmethylamide (CPh—(C₆H₄-4-OMe)₂), N-Bis(4- |
| | methylsulfinylphenyl)methylamide (CH(C₆H₄(O)S-4-Me)₂), N-Triphenylmethylamide (C(C₆H₅)₃), N-9- |
| | Phenylfluorenylamide, N-t-Butoxycarbonylamide (CO—t-OC₄H₉), N-benzyloxycarbonylamide, N- |
| | Methoxycarbonylamide (COOMe), N-Ethoxycarbonylamide (COOEt), N-p-Toluenesulfonylamide, N- |
| | Butenylamide (CH═CHCH₂CH₃), N-[(E)-2-(Methoxycarbonyl)vinyl]amide (CH═CCO₂Me), N- |
| | Diethoxymethylamide (CH(OEt)₂), N-(1-Methoxy-2,2-dimethylpropyl)amide, N-2-(4- |
| | Methylphenylsulfonyl)ethylamide (CH₂CH₂SO₂C₆H₄-4-CH₃) |

Also, lysine, arginine, cysteine, thiolalkylamine, or polyethyleneimine, polylysine, polyarginine, polyaminoester or protamine having various molecular weights (e.g., weight average molecular weight of 100 to 100,000) may be reacted with the polyphosphazene having a carboxylic acid to introduce a functional group to the polyphosphazene.

The phosphazene-based polymer of the present invention has the feature that it contains an ionic group (corresponding to R⁷) and thus can control the degradation rate. As for the ionic group used in the present invention for controlling the degradation rate, the hydrophobicity of the ionic group may be influenced by the structure of the substituent having the ionic group (e.g., NH(R⁶)(R⁷) and NH(R⁶)(R⁷)(R⁸)). In other words, the more hydrophobic structure is more helpful for the polymer to form a hydrophobic network structure and protects the polymer from moisture, whereby physical strength of the gel becomes higher and biodegradation of the gel due to the moisture occurs in a retarded manner. Hydrophobicity of the ionic group can be controlled depending on the carbon number of the dicarboxylic acid-based compound which corresponds to R⁷. That is, the more carbon number of the dicarboxylic acid-based compound gives the higher hydrophobicity, which slows down the degradation rate of the polymer. On the contrary, the less carbon number of the dicarboxylic acid-based compound gives the lower hydrophobicity, which facilitates the degradation of the polymer. Thus, the phosphazene-based polymer of the present invention may be used by suitably selecting the carbon number of the dicarboxylic acid-based compound or the cyclic anhydride compound used for the preparation thereof depending on the purpose of use (e.g., kinds of bioactive substances to be delivered in the body, degradation rate required in the body, etc.). For example, when degradation of the polymer must be retarded, for example, during the delivery of bioactive substances whose sustained release is required in the body, the compound having 5 to 6 carbon atoms (e.g., glutaric anhydride, adipic anhydride, etc.) is employed as the dicarboxylic acid-based compound to make the polymer more hydrophobic and thus to control the degradation slowly. When prompt degradation of the polymer is needed, for example, during the delivery of bioactive substances whose immediate release is required in the body, the compound having 3 to 4 carbon atoms (e.g., maleic anhydride, succinic anhydride, etc.) is employed as the dicarboxylic acid-based compound to make the polymer less hydrophobic and thus to control the degradation promptly.

There is a correlation between the carbon number of the dicarboxylic acid-based compound and the degradation rate of the polymer, and it can be represented by the equation of f(x)=5x−10 (x≧3, carbon number, f(x)=degradation rate (day), the period of time until 50% of the gel is degraded). For example, when glutaric anhydride having 5 carbon atoms is used, 5*5-10=15. Thus, it may be expected that 15 days are needed for the gel to be reduced to 50%. This result may of course be varied depending on the amount of the compound substituted.

In another aspect, the present invention provides a phosphazene-based polymer hydrogel which comprises a solution of the phosphazene-based polymer of Formula (1). The phosphazene-based polymer hydrogel according to the present invention has the characteristics that it shows the conspicuous temperature-dependent sol-gel phase transition, is biodegradable, and has a functional group that can chemically combine with a drug. The phosphazene-based polymer hydrogel may be in the form of a polymer solution wherein the phosphazene-based polymer of Formula (1) is dissolved in one or more suitable solvents selected from the group consisting of water, buffer, acidic solution, basic solution, salt solution, physiological saline, water for injection and dextrose saline in the concentration of 1 to 50 wt %, preferably 3 to 20 wt %.

Since the phosphazene-based polymer and the phosphazene-based polymer hydrogel show the sol-gel phase transition under the temperature ranging from about 5 to 70° C., they may have the gel phase under the body temperature range. Also, they have various functional groups that can combine with various bioactive substances such as drugs, cells, etc., whereby they can be effectively used as a delivery material of various bioactive substances in the body. The phosphazene-based polymer having Formula (1) may have the weight average molecular weight of 4000 to 400000 in order to show excellent sol-gel phase transition and be suitable in bearing the various bioactive substances.

The biodegradable phosphazene-based polymer of Formula (1), which shows the temperature-dependent sol-gel phase transition and has a functional group, may be prepared according to the following method. This method may comprise the following steps of:

(1) subjecting a phosphazene trimer of the following Formula (2)

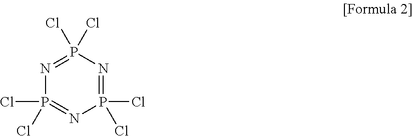 [SMILES: ClP1(Cl)=NP(Cl)(Cl)=NP(Cl)(Cl)=N1]

to the thermal polymerization to obtain a linear polymer of dichlorophosphazene of the following Formula (3)

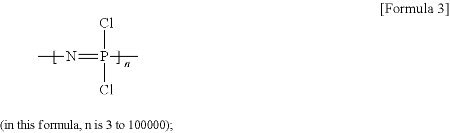

(2) reacting the compound of Formula (3) prepared in Step (1) with an amino acid ester of the following Formula (4)

NH₂CH(R¹)CO₂R² [Formula 4]

or a salt thereof;

(3) reacting the compound prepared in Step (2) with an amino acid, a peptide, a depsipeptide ester or a salt thereof of the following Formula (5)

NH₂CH(R³)(R⁴)(R⁵) [Formula 5]

(4) reacting the compound prepared in Step (3) with a hydroxy group-containing substituent of the following Formula (6)

NH₂(R⁶) [Formula 6]

or a salt thereof;

(5) reacting the compound prepared in Step (4) with aminomethoxy polyethylene glycol of the following Formula (7)

NH₂(CH₂CH₂O)_{P}CH₃ [Formula 7]

or a salt thereof; and

(6) reacting the compound prepared in Step (5) with a cyclic anhydride for forming a compound defined as R⁷.

Moreover, the preparation method of the present invention may further include a step of reacting the carboxylic acid-containing compound prepared in Step (6) with lysine, arginine, cysteine, thiolalkylamine, polyethyleneimine, polylysine, polyarginine or protamine having various molecular weights to prepare the phosphazene-based polymer wherein R⁸ has various functional groups such as NHCH(SH)CO₂H, NH(CH₂)_{q}SH, NH(CH₂CH₂NH)ᵣH, [NHCH(C₄H₈NH₂)CO]ᵣOH, [NHCH[(CH₂)₃C(═NH)(NH₂)]CO]ᵣOH, folic acid, hyaluronic acid, polyhistidine, cyclodextrin, heparin, chitosan, protamine, RGD or RGD derivatives consisting of 4 to 5 amino acids comprising RGD, etc.

The cyclic anhydride compound used in Step (6) may be suitably selected depending on the kind of compound defined as R⁷, for example, from the group consisting of methylsuccinc anhydride, 3-3-dimethylglutaric anhydride, phenylsuccinic anhydride, aconitic anhydride, dimethylmaleic anhydride, itaconic anhydride, diglycolic anhydride, citraconic anhydride, glutaric anhydride, succinic anhydride, maleic anhydride, 2,2-dimethylsuccinic anhydride, 3-methylglutaric anhydride, phenylmaleic anhydride, 2-phenylglutaric anhydride, dodecenylsuccinic anhydride, dimethylmaleic anhydride, N—Z-L-aspartic anhydride, thiodiglycolic anhydride, 2-5-oxazolidindione, tetrafluorosuccinic anhydride, cis-aconitic anhydride, 1-cyclopentene-1,2-dicarboxylic anhydride, phthalic anhydride, 3,6-dichlorophthalic anhydride and adipic anhydride.

The above method for preparing the functional group-containing phosphazene-based polymer of Formula (1) may be summarized as the following Reaction Scheme (1):

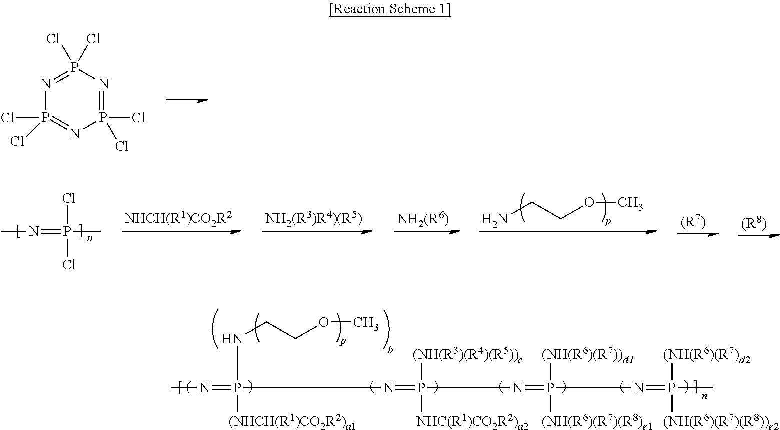

in the above Formulas (4), (5), (6), (7) and Reaction Scheme (1), R¹, R², R³, R⁴, R⁵, R⁶, R⁷, R⁸, a₁, a₂, b, c, d₁, d₂, e₁, e₂, n and p are as defined in Formula (1).

The method for preparing the functional group-containing phosphazene-based polymer of Formula (1) will be explained more in detail below. All the preparation processes are preferably carried out under vacuum and/or nitrogen line in order to exclude moisture. It is desirable to sufficiently remove the moisture from various solvents used in the reactions by conventional methods before they are used.

Step (1) may be performed by introducing the compound of Formula (2) and 0.1 to 10 wt % of AlCl₃ into a glass reaction tube, sealing the tube and reacting them at 200 to 250° C. for 4 to 8 h while swirling the tube at a speed of 1 rpm.

Step (2) may be performed by reacting 1 eq. of the compound prepared in Step (1) under the presence of 0.01 to 1.9 eq. of the amino acid ester of Formula (4) or a salt thereof and 4 eq. of triethylamine. It is preferable that the salt of the amino acid ester of Formula (4) is hydrochloride or sulfate. The reaction solvent may be selected from the group consisting of tetrahydrofuran (THF), dioxane, chloroform and toluene, but is not limited thereto. The reaction may be performed at −60 to 50° C. for about 8 to 72 h.

Step (3) may be performed by reacting the compound prepared in Step (2) under the presence of 0 to 1.9 eq. of the amino acid, peptide or depsipeptide ester of Formula (5) or a salt thereof and 4 eq. of triethylamine. It is preferable that the salt of the compound of Formula (5) is oxalate, hydrochloride or trifluoroacetate. The reaction solvent may be selected from the group consisting of acetonitrile, tetrahydrofuran, dioxane, chloroform and toluene, but is not limited thereto. The reaction may be performed at 0 to 50° C. for about 1 to 72 h.

Step (4) may be performed by reacting the compound prepared in Step (3) under the presence of 0.01 to 1.9 eq. of the functional group-containing substituent of Formula (6) or a salt thereof and 4 eq. of triethylamine. It is preferable that the salt of the compound of Formula (6) is oxalate, hydrochloride or trifluoroacetate. The reaction solvent may be selected from the group consisting of acetonitrile, tetrahydrofuran, dioxane, chloroform and toluene, but is not limited thereto. The reaction may be performed at 25 to 50° C. for about 12 to 72 h.

Step (5) may be performed by reacting the compound prepared in Step (4) under the presence of 2 eq. (based on the amount of remaining chlorine) of aminomethoxy polyethylene glycol of Formula (6) and 4 eq. of triethylamine to substitute all the remaining chlorine in the product of Step (4). The reaction solvent may be selected from the group consisting of tetrahydrofuran, dioxane, chloroform and toluene, but is not limited thereto. The reaction may be performed at 25 to 50° C. for about 24 to 72 h.

Step (6) may be performed by an esterification reaction using 1 to 5 mol of the cyclic anhydride which is suitable for the desired dicarboxyl-based compound of R⁷ under the presence of 1 to 5 mol of 4-dimethylaminopyridine to produce an ester carboxylic group. In this case, the reaction solvent may be selected from the group consisting of tetrahydrofuran and dioxane, but is not limited thereto. The reaction may be preferably performed at 20 to 50° C. for about 1 to 48 h.

Step (7) is performed by reacting the carboxylic acid-containing compound prepared in Step (5) or (6) in the presence of lysine, arginine, histidine, cysteine, thiolalkylamine (e.g., having 1 to 50 carbon atoms), or polyethyleneimine, polylysine or polyarginine having various weight average molecular weights, folic acid, hyaluronic acid, polyhistidine, imidazole-based compound, spermine, spermidine, cyclodextrin, heparin, chitosan or protamine, 1 to 3 eq. of dicyclohexylcarbodiimide and 1 to 3 eq. of hydroxysuccinimide to produce phosphazene-based polymers having various functional groups. The reaction solvent may be selected from the group consisting of tetrahydrofuran and chloroform, but is not limited thereto. The reaction may be preferably performed at 0 to 25° C. for about 1 to 48 h.

In the above Steps (1) to (6), the product of each step may be used per se in the next step without purification. The desired pure product may be recovered from the reaction mixture of Steps (5), (6) and (7) according to the following purification method.

First, the reaction mixture is centrifuged or filtered to remove the precipitate (e.g., triethylammonium chloride, triethylammonium salt of oxalic acid, etc.) therefrom, and the filtrate is concentrated under reduced pressure until only a little amount of solvent remains. The concentrate thus obtained is dissolved in tetrahydrofuran, and an excess amount of ethyl ether, hexane or a mixture of ethyl ether and hexane is added thereto to induce precipitation of the product. The precipitate is filtered twice or three times to remove the unreacted substituents. The compound obtained through these processes is dissolved again in a small amount of methyl alcohol or ethyl alcohol, dialyzed with methyl alcohol or ethyl alcohol at 25° C. for 3 to 10 days and with distilled water at 4 to 25° C. for 3 to 10 days, and dried at a low temperature to obtain the pure compound of Formula (1).

In another aspect, the present invention provides a bioactive substance delivery composition, which comprises one or more selected from the group consisting of the phosphazene-based polymer of Formula (1) and the hydrogel containing a solution of the phosphazene-based polymer. In addition, the present invention provides a bioactive substance delivery system, which comprises one or more selected from the group consisting of the phosphazene-based polymer of Formula (1) and the hydrogel containing a solution of the phosphazene-based polymer, and one or more bioactive substances.

In another aspect, the present invention provides a method for delivery of bioactive substances, which comprises the steps of preparing the bioactive substance delivery system comprising one or more selected from the group consisting of the phosphazene-based polymer of Formula (1) and the hydrogel containing a solution of the phosphazene-based polymer, and one or more bioactive substances, and administering the bioactive substance delivery system to a patient in need of the administration of the bioactive substances. The patient may be mammals including human being, or cells or tissues separated from their bodies. The administration may be performed orally or via parenteral routes such as subcutaneous, intravenous, intramuscular, intrathoracic injection, etc.

As stated above, the phosphazene-based polymer and the phosphazene-based polymer hydrogel according to the present invention have the characteristics that they show the conspicuous temperature-dependent sol-gel phase transition, are biodegradable, and have a functional group that can chemically combine with various bioactive substances such as drugs. In other words, since the phosphazene-based polymer and the phosphazene-based polymer hydrogel show the sol-gel phase transition under the temperature ranging from about 5 to 70° C., they may have the gel phase under the body temperature range. Also they have various functional groups that can combine with various bioactive substances such as drugs, cells, etc., whereby they can be effectively used as a delivery material of various bioactive substances in the body.

The phosphazene-based polymer having Formula (1) may have the molecular weight of 4000 to 400000 in order to show excellent sol-gel phase transition and be suitable in bearing the various bioactive substances.

If the phosphazene-based polymer or the phosphazene-based polymer hydrogel bearing various bioactive substances such as drugs, therapeutic cells, etc. is introduced into the body, it forms a gel phase having a three-dimensional structure at body temperature, and the bioactive substances chemically combine with the functional group of the phosphazene-based polymer. Thereby, it can prevent the initial burst release of the bioactive substances in the body and control the release rate to make the sustained and effective release possible. In particular, since the degradation rate should be different depending on the characteristics of drugs, in order to avoid the inconvenience of considering the degradation rate from the first stage of manufacturing process, the kind of ionic group substituted may be selected to control the degradation rate even after the main synthetic reaction is performed. Thus, the phosphazene-based polymer or the phosphazene-based polymer hydrogel is very suitable as a composition for delivering various bioactive substances in the body. The phosphazene-based polymer or hydrogel may be particularly useful for the delivery of proteins having various charges and molecular weights.

The bioactive substances to be delivered by the bioactive substance delivery composition of the present invention and those contained in the bioactive substance delivery system may be any substance showing profitable in vivo effect, for example, one or more selected from the group consisting of drug and therapeutic cell. The drug may be one or more selected from the group consisting of protein, polypeptide, peptide, vaccine, gene, hormone, anticancer agent and angiogenesis inhibitor.

The protein, polypeptide and peptide may be one or more selected from the group consisting of exendin-4, erythropoietin (EPO), interferon-alpha, interferon-beta, interferon-gamma, growth hormone (human, pig, cow, etc.), growth hormone releasing factor, nerve growth factor (NGF), G-CSF (granulocyte-colony stimulating factor), GM-CSF (granulocyte macrophage-colony stimulating factor), M-CSF (macrophage-colony stimulating factor), blood clotting factor, insulin, oxytocin, basopressin, adrenocorticotropic hormone, fibroblast growth factor (FGF), epidermal growth factor (EGF), platelet-derived growth factor (PDGF), insulin-like growth factor (IGF), vascular endothelial growth factor (VEGF), transforming growth factor-beta (TGF-β), brain-derived neurotrophic factor (BDNF), neurotrophin-3 (NT-3), neurotrophin-4/5, prolactin, luliberin, luteinizing hormone releasing hormone (LHRH), LHRH agonists, LHRH antagonists, somatostatin, glucagon, interleukin-2 (IL-2), interleukin-11 (IL-11), gastrin, tetragastrin, pentagastrin, urogastrone, secretin, calcitonin, enkephalins, endorphins, angiotensins, thyrotropin releasing hormone (TRH), tumor necrosis factor (TNF), tumor necrosis factor related apoptosis inducing ligand (TRAIL), heparinase, bone morphogenic protein (BMP), hANP (human atrial natriuretic peptide), glucagon-like peptide (GLP-1), renin, bradykinin, bacitracins, polymyxins, colistins, tyrocidine, gramicidins, cyclosporins, neurotensin, tachykinin, neuropeptide Y (NPY), peptide YY (PYY), vasoactive intestinal polypeptide (VIP), pituitray adenylate cyclase-activating polypeptide (PACAP); synthetic analogs thereof, antibodies (e.g., monoclonal antibody or polyclonal antibody), and moieties which are modified or show the same efficacy as the drug; enzymes; and cytokines.

The vaccine may be one or more selected from the group consisting of hepatitis vaccine and the like.

The gene may be one or more selected from the group consisting of small interference RNA (siRNA), plasmid DNA, antisense oligodeoxynucleotide (AS-ODN), etc.

The hormone may be one or more selected from the group consisting of testosterone, estradiol, progesterone, prostaglandins, synthetic analogs thereof, and substances which are modified or show the same efficacy as the hormone.

The anticancer agent may be one or more selected from the group consisting of paclitaxel, doxorubicin, 5-fluorouracil, cisplatin, carboplatin, oxaliplatin, tegafur, irinotecan, docetaxel, cyclophosphamide, cemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansine, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassuim, medroxypogesterone acetate, epirubicin, letrozole, pirarubicin, topotecan, altretamine, toremifene citrate, BCNU, taxotere, actinomycin D, anasterozole, belotecan, imatinib, floxuridine, gemcitabine, hydroxyurea, zoledronate, vincristine, flutamide, valrubicin, streptozocin, polyethylene glycol-conjugated anticancer agents wherein polyethylene glycol is conjugated to the above anticancer agent, synthetic analogs thereof, and substances which are modified or show the same efficacy as the anticancer agent.

The angiogenesis inhibitor may be one or more selected from the group consisting of clodronate, 6-deoxy-6-demethyl-4-dedimethylaminotetracycline (COL-3), doxycycline, marimastat, 2-methoxyestradiol, squalamine, thalidomide, TNP-470, combretastatin A4, soy isoflavone, enzastaurin, revimid (e.g., CC 5013, Celgene Corp, Warren, N.J.), celecoxib, vandetanib (e.g., ZD 6474), halofuginone hydrobromide, interferon-alpha, bevacizumab, shark cartilage extract (e.g., AE-941), interleukin-12, vascular endothelial growth factor trap (VEFG-trap), cetuximab, rebimastat, matrix metalloproteinase (MMP) inhibitor (e.g., BMS-275291 (Bristol-Myers Squibb, New York, N.Y.), S-3304, etc.), protein kinase C beta inhibitor (e.g., LY317615), endostatin, vatalanib (e.g., PTK787/ZK 222584), sunitinib malate (e.g., SU11248), cilenqitide (e.g., EMD-121974), humanized monoclonal antibody (e.g., MEDI-522), volociximab (e.g., EOS-200-4), integrin alpha-5-beta-1 antagonists (e.g., ATN-161), synthetic analogs thereof, and substances which are modified or show the same efficacy as the angiogenesis inhibitor.

The therapeutic cell may be one or more selected from the group consisting of preosteoblast, chondrocyte, umbilical vein endothelial cell (UVEC), osteoblast, adult stem cell, schwann cell, oligodendrocyte, hepatocyte, mural cell (used in combination with UVEC), myoblast, insulin secreting cell, endothelial cell, smooth muscle cell, fibroblast, P3 cell, endodermal cell, hepatic stem cell, juxraglomerular cell, skeletal muscle cell, keratinocyte, melanocyte, langerhans cell, merkel cell, dermal fibroblast, and preadipocyte.

In case that the bioactive substance delivery system of the present invention contains a drug as the bioactive substance, the content of the drug is desirably from 1×10⁻⁸ to 50 vol %, preferably 1×10⁴ to 20 vol %, based on the total volume of the bioactive substance delivery system. Also, in case that the bioactive substance delivery system of the present invention contains a cell as the bioactive substance, the content of the cell is desirably from 1×10⁻⁸ to 50 vol % based on the total volume of the bioactive substance delivery system. If the content of the drug or cell is lower than said ranges, the desired effect of the drug cannot be obtained. On the other hand, if the content exceeds said ranges, the property of the polymer may be deteriorated.

The bioactive substance delivery system comprising the phosphazene-based polymer of Formula (1) or the phosphazene-based polymer hydrogel according to the present invention may further comprise the additives as described below. The bioactive substance delivery system comprising the phosphazene-based polymer or the phosphazene-based polymer hydrogel according to the present invention may further comprise various additives, thereby the efficacy of the polymer hydrogel as a material for delivery of bioactive substances such as drugs may be increased. For example, the sol-gel phase transition of the aqueous phosphazene-based polymer solution may be controlled by the addition of various salts to achieve the desired gel solidity and gelling temperature (Macromolecules 32, 7820, 1999). When a polypeptide or protein drug is to be delivered, the introduction of proper additives allows the stability of the drug in the hydrogel to be maintained. Furthermore, a chemical bond including an ionic bond, etc. between the drug and additive may be induced to control the release rate of the drug from the hydrogel. Moreover, when a therapeutic cell is to be delivered, the activity of the cell after delivery into the body may be increased due to the additives introduced to the hydrogel.

That is, the additives may induce various interactions for chemical bonds including an ionic bond between the phosphazene-based polymer or the phosphazene-based polymer hydrogel and the bioactive substance including a drug, to control the release of the bioactive substances and/or increase the in vivo activity of the bioactive substance including a therapeutic cell.

In the present invention, the content of the additive may be from 1×10⁻⁶ to 30 wt %, more preferably 1×10⁻³ to 10 wt %, based on the total weight of the bioactive substance delivery composition or the bioactive substance delivery system. If the content of the additive is lower than said range, the effect desired by the additive cannot be obtained. On the other hand, if the content exceeds said range, the effect of the active ingredient and/or the physical property of the thermosensitive polymer according to the present invention may be deteriorated.

The additive may be any substance inducing various interactions between the phosphazene-based polymer and the bioactive substance, for example, one or more selected from the group consisting of cationic polymer having the weight average molecular weight of 200 to 750,000, anionic polymer having the weight average molecular weight of 200 to 750,000, amino acid, peptide, protein, fatty acid, phospholipid, vitamin, drug, polyethylene glycol ester (e.g., those having the weight average molecular weight of 300 to 50,000), steroid, amine compound, acrylic copolymer (e.g., those having the weight average molecular weight of 300 to 500,000), organic solvent, preservative, sugar, polyol, sugar-containing polyol, sugar-containing amino acid, surfactant, sugar-containing ion, silicate, metal salt and ammonium salt.

More specifically, the additive may be one or more selected from the group consisting of as cationic polymers (for example, those having a molecular weight of 200 to 750,000) such as poly-L-arginine, poly-L-lysine, poly(ethylene glycol), polyethylenimine, chitosan, protamin, etc.; anionic polymers such as polyvinylacetate (PVA), hyaluronic acid, chondroitin sulphate, heparin, alginate, etc.; growth factors such as amiloride, procainamide, acetyl-beta-methylcholine, spermine, spermidine, lysozyme, fibroin, albumin, collagen, transforming growth factor-beta (TGF-beta), fibroblast growth factor (bFGF), vascular endothelial growth factor (VEGF), etc.; bio-materials such as bone morphogenetic proteins (BMPs), dexamethason, fibronectin, fibrinogen, thrombin, protein, dexrazoxane, leucovorin, ricinoleic acid, phospholipid, small intestinal submucosa, vitamin E, polyglycerol ester of fatty acid, labrafil, citric acid, glutamic acid, hydroxypropyl methylcellulose, gelatin, isopropyl myristate, eudragit, tego betain, dimyristoylphosphatidylcholine, scleroglucan, etc; organic solvents such as chromophore EL, ethanol, dimethylsulfoxide, etc.; preservatives such as methylparaben, etc.; saccharides such as starch, cyclodextrin and its derivatives, lactose, glucose, dextran, mannose, sucrose, trehalose, maltose, ficoll, etc.; polyols such as inositol, mannitol, sorbitol, etc.; sugar-containing polyols such as sucrose-mannitol, glucose-mannitol, etc.; amino acids such as alanine, arginine, glycine, etc.; sugar-containing polyols such as trehalose-PEG, sucrose-PEG, sucrose-dextran, etc.; sugar-containing amino acids such as sorbitol-glycine, sucrose-glycine, etc.; surfactants such as poloxamers having various molecular weights, Tween 20, Tween 80, Triton X-100, sodium dodecyl sulfate (SDS), Brij, etc.; sugar-containing ions such as trehalose-ZnSO₄, maltose-ZnSO₄, etc.; and salts such as silicate, NaCl, KCl, NaBr, NaI, LiCl, n-Bu₄NBr, n-Pr₄NBr, Et₄NBr, Mg(OH)₂, Ca(OH)₂, ZnCO₃, Ca₃(PO₄)₂, ZnCl₂, (C₂H₃O₂)₂Zn, ZnCO₃, CdCl₂, HgCl₂, CoCl₂, (CaNO₃)₂, BaCl₂, MgCl₂, PbCl₂, AlCl₃, FeCl₂, FeCl₃, NiCl₂, AgCl, AuCl₃, CuCl₂, sodium dodecyl sulfate, sodium tetradecyl sulfate, dodecyltrimethylammonium bromide, dodecyltrimethylammonium chloride, tetradecyltrimethylammonium bromide, etc.

Hereinafter, the present invention will be explained more in detail by the Examples. However, the following Examples are only for the illustration of the present invention and it is not intended that the scope of the present invention is limited in any manner by them.

The phosphazene-based polymer for drug delivery, which contains a degradation controllable ionic group, according to the present invention can make a direct chemical bond or an ionic bond with a drug and thus be used as a drug delivery material capable of releasing the drug. At the same time, it can easily control the degradation rate by the selection of the ionic group to induce the disappearance of gel within the desired period of time. Thus, it is expected to be applicable in various industrial fields including drug delivery and tissue engineering.

### BRIEF DESCRIPTION OF THE DRAWINGS

FIG. 1 is a photograph showing the sol-gel phase transition of the polyphosphazene-based polymer for drug delivery which contains a degradation controllable ionic group of the present invention.

FIG. 2 shows the temperature-dependent viscosity change of the polyphosphazene-based polymer for drug delivery used in the present invention.

FIG. 3 shows the temperature-dependent viscosity change of the polymer, varying according to two kinds of ionic groups which are different from each other and are introduced to the same phosphazene polymer of the present invention.

FIG. 4 shows the time-dependent decrease of the amount of gel of the polymer, observed by the naked eye, varying according to the kind of degradation controllable ionic group of the present invention.

FIG. 5 shows the time-dependent decrease of the weight of gel of the polymer, varying according to the kind of degradation controllable ionic group of the present invention.

FIG. 6 shows the release behavior of the human growth hormone from the polymer with lapse of time, varying according to the kind of degradation controllable ionic group of the present invention.

### EXAMPLE

In the following Examples, the elementary analysis of carbon, hydrogen and nitrogen for the product was performed by the Property Analysis Center in the Korea Advanced Institute of Science and Technology using the Perkin-Elmer C, H, N analyzer. The nuclear magnetic resonance spectrums with hydrogen and phosphorus were respectively measured by using Varian Gemini-300, and the weight average molecular weight (M_{w}) was measured through gel permeation chromatography using a Waters 1515 pump and a 2410 differentiation refractomer. Also, the nomenclature of Examples was based on the abbreviations described in the following Table 3.

TABLE 3

| | | Selected | | |
|---|---|---|---|---|
| Substituent | Kind | Substituent | Formula | Abbreviation |
| NH(R¹)CO₂R² | R¹ | CH(CH₃)C₂H₅ | NHCH(CH₃)(C₂H₅)CO₂(C₂H₅) | I |
| | R² | C₂H₅ | | |
| NH(CH₂CH₂O)ₚCH₃ | p = 16 | | NH(CH₂CH₂O)ₙCH₃ | P |
| NH(R³)(R⁴)(R⁵) | — | — | — | — |
| NH(R⁶)(R⁷) and/or | R⁶ | (CH₂)ₗOH, l = 2 | NH(CH₂)₂OH | H1 |
| NH(R⁶)(R⁷)(R⁸) | | | (Form in the compound: NH(CH₂)₂O−) | |
| | | (CH₂)ₗOH, l = 3 | NH(CH₂)₃OH | H2 |
| | | | (Form in the compound: NH(CH₂)₃O−) | |
| | | (CH₂)ₗOH, l = 5 | NH(CH₂)₅OH | H3 |
| | | | (Form in the compound: NH(CH₂)₅O−) | |
| | R⁷ | CO(CH₂)ₘCOOH, | NH(CH₂)₂OCO(CH₂)₂COOH | H1A1 |
| | | m = 2 | (Form in the compound in the presence | |
| | | | of R⁸: NH(CH₂)₂OCO(CH₂)₂COO−) | |
| | | CO(CH₂)ₘCOOH, | NH(CH₂)₃OCO(CH₂)₂COOH | H2A1 |
| | | m = 2 | (Form in the compound in the presence | |
| | | | of R⁸: NH(CH₂)₃OCO(CH₂)₂COO−) | |
| | | CO(CH₂)ₘCOOH, | NH(CH₂)₂OCO(CH₂)₃COOH | H1A2 |
| | | m = 3 | (Form in the compound in the presence | |
| | | | of R₈: NH(CH₂)₂OCO(CH₂)₃COO−) | |
| | | CO(CH₂)ₘCOOH, | NH(CH₂)₂OCO(CH₂)₄COOH | H1A3 |
| | | m = 4 | (Form in the compound in the presence | |
| | | | of R⁸: NH(CH₂)₂OCO(CH₂)₄COO−) | |
| | R⁸ | Polyethylene- | NH(CH₂)₂OCO(CH₂)₂CONH-PEI | H1A1PEI |
| | | imine | | |
| | | Protamine | NH(CH₂)₂OCO(CH₂)₂CONH-Pro | H1A1Pro |
| | | Imidazole | NH(CH₂)₂OCO(CH₂)₂CONH-Imi | H1A1Imi |

### Example 1

### [NP(I)_{1.22}(P)_{0.57}(H1)_{0.21}]

Poly(dichlorophosphazene) (9.00 g, 77.66 mmol) was dissolved in tetrahydrofuran (300 ml), isoleucine ethyl ester hydrochloride (21.50 g, 109.89 mmol) and triethylamine (53.61 ml, 384.61 mmol) were added in the order in a dryice-acetone bath, and the mixture was reacted at room temperature for 48 h. To the reaction solution thus obtained was added a tetrahydrofuran solution (50 ml) wherein ethanolamine (1.40 g, 23.30 mmol) and triethylamine (6.49 ml, 46.60 mmol) were dissolved. Then, a tetrahydrofuran solution (100 ml) wherein aminomethoxy polyethylene glycol having the molecular weight of 750 (16.44 g, 15.53 mmol) and triethylamine (13.98 g, 69.04 mmol) were dissolved was added thereto and reacted at room temperature for 48 h. Subsequently, a tetrahydrofuran solution (50 ml) wherein aminomethoxy polyethylene glycol having the molecular weight of 750 (8.22 g, 7.76 mmol) and triethylamine (6.99 g, 34.52 mmol) were dissolved was further added thereto and reacted at room temperature for 48 h. The reaction solution was filtered to remove the resulting triethylamine hydrochloride. The filtrate was concentrated under reduced pressure until only a small amount of solvent remained. The concentrate thus obtained was dissolved in tetrahydrofuran (100 ml), and excess hexane was added to induce precipitation. This process was repeated twice or three times.

The resulting precipitate was dissolved again in a small amount of methyl alcohol, dialyzed with methyl alcohol at room temperature for 5 days and with distilled water for 5 days, and dried at a low temperature to give the final product [NP(I)_{1.22}(P)_{0.57}(H1)_{0.21}]ₙ (17.61 g, Yield 80%).

Hydrogen Nuclear Magnetic Resonance Spectrum (CDCl₃, ppm):

δ 0.7˜1.1 (b, —NHCH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ 2.67˜3.2 (b, —NHCH₃CH₂OH, —NH(CH₂CH₂O)₁₆CH₃),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃)

Average molecular weight (M_{w}): 14000

### Example 2

### [NP(I)_{1.18}(P)_{0.42}(H2)_{0.40}]ₙ

Poly(dichlorophosphazene) (9.00 g, 77.66 mmol), isoleucine ethyl ester (20.21 g, 103.29 mmol), aminomethoxy polyethylene glycol having the molecular weight of 750 (66.40 g, 88.53 mmol), propanolamine (0.58 g, 7.77 mmol), triethylamine (120.74 ml, 1.64 mol) and tetrahydrofuran (600 ml) were used according to the same procedure as Example 1 to give the final product [NP(I)_{1.18}(P)_{0.42}(H2)_{0.40}]ₙ (16.54 g, Yield 77%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃, b, —NHCH₂CH₂CH₂OH),

δ 2.67˜3.2 (b, —NHCH₂CH₂CH₂OH, —NH(CH₂CH₂O)₁₆CH₃),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃)

Average molecular weight (M_{w}): 15000

### Example 3

### [NP(I)_{1.36}(P)_{0.41}(H3)_{0.24}]ₙ

Poly(dichlorophosphazene) (2.00 g, 17.26 mmol), isoleucine ethyl ester (3.61 g, 18.47 mmol), pentanolamine (1.07 g, 10.35 mmol), aminomethoxy polyethylene glycol having the molecular weight of 750 (8.54 g, 11.38 mmol), triethylamine (16.37 g, 118.14 mmol) and tetrahydrofuran (400 ml) were used according to the same procedure as Example 1 to give the final product [NP(I)_{1.18}(P)_{0.42}(H3)_{0.40}]ₙ (5.37 g, Yield 76%).

Hydrogen Nuclear Magnetic Resonance Spectrum (CDCl₃, ppm):

δ 0.7˜1.1 (b, —NHCH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),
b, —NHCH₂CH₂CH₂CH₂CH₂OH),

δ 2.67˜3.2 (b, —NHCH₂CH₂CH₂CH₂CH₂OH, —NH(CH₂CH₂O)₁₆CH₃),

δ 3.4 (s, —NH(CH₂CH₂O)₁₆CH₃),

δ 3.4˜3.8 (b, —NH(CH₂CH₂O)₁₆CH₃, b, —NHCH₂CH₂CH₂CH₂CH₂OH),

δ 3.9˜4.3 (b, —NHCH(CH(CH₃)CH₂CH₃)COOCH₂CH₃)

Average molecular weight (M_{w}): 15500

### Example 4

### [NP(I)_{1.32}(P)_{0.42}(H1A1)_{0.36}]ₙ

Poly(dichlorophosphazene) (10.00 g, 86.29 mmol), isoleucine ethyl ester (22.46 g, 114.76 mmol), ethanolamine (2.21 g, 36.24 mmol), aminomethoxy polyethylene glycol having the molecular weight of 750 (18.08 g, 24.10 mmol), triethylamine (84.35 ml, 593.2 mmol) and tetrahydrofuran (700 ml) were used according to the same procedure as Example 1 to give the final product [NP(I)_{1.42}(P)_{0.21}(H1)_{0.37}]ₙ (30 g, Yield 92%). Subsequently, [NP(I)_{1.42}(P)_{0.21}(H1)_{0.37}]ₙ (25 g) thus obtained was dissolved in tetrahydrofuran (500 ml) and reacted with 2 eq. of succinic anhydride (2.95 g) and 2 eq. of dimethylaminopyridine (3.60 g) at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the final product [NP(I)_{1.32}(P)_{0.42}(H1A 1)_{0.36}]ₙ (26 g, Yield 81%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ 2.5˜2.7 (b, —NHCH₂CH₂OCOCH₂CH₂COOH),

δ 2.9˜3.2 (b, —NHCH₂CH₂OCOCH₂CH₂COOH),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃,
—NHCH₂CH₂OCOCH₂CH₂COOH)

Average molecular weight (M_{w}): 15000

### Example 5

### [NP(I)_{1.32}(P)_{0.42}(H2A1)_{0.36}]ₙ

Poly(dichlorophosphazene) (9.00 g, 77.66 mmol), isoleucine ethyl ester (20.21 g, 103.29 mmol), aminomethoxy polyethylene glycol having the molecular weight of 750 (66.40 g, 88.53 mmol), propanolamine (0.58 g, 7.77 mmol), triethylamine (120.74 ml, 1.64 mol) and tetrahydrofuran (600 ml) were used according to the same procedure as Example 1 to give the final product (16.54 g, Yield 77%). Subsequently, [NP(I)_{1.18}(P)_{0.42}(H2)_{0.40}]ₙ (10 g) thus obtained was dissolved in tetrahydrofuran (500 ml) and reacted with 2 eq. of succinic anhydride (1.04 g) and 2 eq. of dimethylaminopyridine (1.27 g) at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the final product [NP(I)_{1.32}(P)_{0.42}(H1A1)_{0.36}]ₙ (11 g, Yield 81%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),
(b, —NHCH₂CH₂CH₂OCOCH₂CH₂COOH),

δ 2.5˜2.7 (b, —NHCH₂CH₂CH₂OCOCH₂CH₂COOH),

δ 2.9˜3.2 (b, —NHCH₂CH₂CH₂OCOCH₂CH₂COOH),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃),
(b, —NHCH₂CH₂CH₂OCOCH₂CH₂COOH),

Average molecular weight (M_{w}):

### Example 6

### [NP(I)_{1.48}(P)_{0.41}(H1A2)_{0.11}]ₙ

Poly(dichlorophosphazene) (10.00 g, 86.29 mmol), isoleucine ethyl ester (23.13 g, 118.21 mmol), ethanolamine (1.58 g, 25.89 mmol), aminomethoxy polyethylene glycol having the molecular weight of 750 (24.59 g, 32.79 mmol), triethylamine (80.89 ml, 580.28 mmol) and tetrahydrofuran (700 ml) were used according to the same procedure as Example 1 to give the final product [NP(I)_{1.48}(P)_{0.10}(H1)_{0.42}]ₙ (32 g, Yield 92%). Subsequently, [NP(I)_{1.48}(P)_{0.10}(H1)_{0.42}]ₙ (15 g) thus obtained was dissolved in tetrahydrofuran (200 ml). To this solution were added the tetrahydrofuran solution (50 ml) wherein 2 eq. of glutaric anhydride (0.51 g) was dissolved and the tetrahydrofuran solution (50 ml) wherein 2 eq. of dimethylaminopyridine (0.55 g) was dissolved, and the whole mixture was reacted at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the final product [NP(I)_{1.48}(P)_{0.41}(H1A2)_{0.11}]ₙ (14 g, Yield 80%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ2.5˜2.7 (b, —NHCH₂CH₂OCOCH₂CH₂CH₂COOH),

δ 2.9˜3.2 (b, —NHCH₂CH₂OCOCH₂CH₂CH₂COOH),

δ 3.4 (s, —NH(CH₂CH₂O)₁₆CH₃),

δ 3.4˜3.8 (b, —NH(CH₃CH₂O)₁₆CH₃),

δ 3.9˜4.3 (b, —NHCH(CH(CH₃)CH₂CH₃)COOCH₂CH₃,
—NHCH₂CH₂OCOCH₂CH₂CH₂COOH)

Average molecular weight (M_{w}): 11000

### Example 7

### [NP(I)_{1.48}(P)_{0.33}(H1A2)_{0.19}]ₙ

Poly(dichlorophosphazene) (10.00 g, 86.29 mmol), isoleucine ethyl ester (24.06 g, 122.96 mmol), ethanolamine (1.56 g, 25.89 mmol), aminomethoxy polyethylene glycol having the molecular weight of 750 (26.7 g, 35.60 mmol), triethylamine (78.78 ml, 565.18 mmol) and tetrahydrofuran (700 ml) were used according to the same procedure as Example 1 to give the final product [NP(I)_{1.45}(P)_{0.38}(H1A2)_{0.17}]ₙ (28 g, Yield 90%). Subsequently, [NP(I)_{1.45}(P)_{0.38}(H1A2)_{0.17}]ₙ (10 g) thus obtained was dissolved in tetrahydrofuran (200 ml). To this solution were added the tetrahydrofuran solution (50 ml) wherein 2 eq. of glutaric anhydride (1.13 g) was dissolved and the tetrahydrofuran solution (50 ml) wherein 2 eq. of dimethylaminopyridine (1.38 g) was dissolved, and the whole mixture was reacted at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the final product [NP(I)_{1.48}(P)_{0.33}(H1A2)_{0.19}]ₙ (14 g, Yield 80%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ 2.5˜2.7 (b, —NHCH₂CH₂OCOCH₂CH₂CH₂COOH),

δ 2.9˜3.2 (b, —NHCH₂CH₂OCOCH₂CH₂CH₂COOH),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃,
—NHCH₂CH₂OCOCH₂CH₂CH₂COOH)

Average molecular weight (M_{w}): 11000

### Example 8

### [NP(I)_{1.53}(P)_{0.42}(H1A3)_{0.05}]ₙ

Poly(dichlorophosphazene) (10.00 g, 86.29 mmol), isoleucine ethyl ester (23.13 g, 118.21 mmol), ethanolamine (1.58 g, 25.89 mmol), aminomethoxy polyethylene glycol having the molecular weight of 750 (24.59 g, 32.79 mmol), triethylamine (80.89 ml, 580.28 mmol) and tetrahydrofuran (700 ml) were used according to the same procedure as Example 1 to give the final product [NP(I)_{1.48}(P)_{0.10}(H1)_{0.42}]ₙ (32 g, Yield 92%). Subsequently, [NP(I)_{1.48}(P)_{0.10}(H1)_{0.42}]ₙ (15 g) thus obtained was dissolved in tetrahydrofuran (200 ml). To this solution were added the methyl chloride solution (40 ml) wherein 2 eq. of adipic anhydride (0.58 g) was dissolved and the methyl chloride solution (40 ml) wherein 2 eq. of dimethylaminopyridine (0.55 g) was dissolved, and the whole mixture was reacted at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the final product [NP(I)_{1.53}(P)_{0.42}(H1A3)_{0.05}]ₙ (10 g, Yield 72%).

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ 2.5˜2.7 (b, —NHCH₂CH₂OCOCH₂CH₂CH₂CH₂COOH),

δ 2.9˜3.2 (b, —NHCH₂CH₂OCOCH₂CH₂CH₂CH₂COOH),

δ 3.4 (s, —NH(CH₂CH₂O)₁₆CH₃),

δ 3.4˜3.8 (b, —NH(CH₂CH₂O)₁₆CH₃),

δ 3.9˜4.3 (b, —NHCH(CH(CH₃)CH₂CH₃)COOCH₂CH₃,
—NHCH₂CH₂OCOCH₂CH₂CH₂CH₂COOH)

Average molecular weight (M_{w}): 11000

### Example 9

### [NP(I)_{1.43}(P)_{0.37}(H1A1)_{0.16}(PEI)_{0.04}]ₙ

Poly(dichlorophosphazene) (4.00 g, 34.52 mmol), isoleucine ethyl ester (8.98 g, 45.91 mmol), ethanolamine (0.63 ml, 10.35 mmol), aminomethoxy polyethylene glycol having the molecular weight of 750 (9.58 g, 12.77 mmol), triethylamine (39.87 ml, 286.2 mmol) and tetrahydrofuran (500 ml) were used according to the same procedure as Example 1 to give the final product [NP(I)_{1.42}(P)_{0.21}(H1)_{0.37}]ₙ (30 g, Yield 92%). Subsequently, [NP(I)_{1.42}(P)_{0.21}(H1)_{0.37}]ₙ (4 g) thus obtained was dissolved in tetrahydrofuran (200 ml) and reacted with 2 eq. of succinic anhydride (0.53 g) and 2 eq. of dimethylaminopyridine (0.53 g) at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the product [NP(I)_{1.43}(P)_{0.37}(H1A1)_{0.20}]ₙ (4.3 g, Yield 91%). Subsequently, [NP(I)_{1.43}(P)_{0.37}(H1A1)_{0.20}]ₙ (3 g) was dissolved in chloroform, to which were added isobutylchloroformate (0.13 ml, 1.03 mmol) and triethyleneamine (0.29 ml, 2.07 mmol), which was then activated for 40 min. Then, polyethyleneimine (9.30 g, 5.17 mmol) was dissolved in chloroform and reacted with the above solution. After 18 h, the reaction mixture was concentrated under reduced pressure and the precipitate was removed by KF solution. The resulting solution was dialyzed with distilled water at 4° C. for 3 days and dried at a low temperature to give the final product [NP(I)_{1.43}(P)_{0.37}(H1A 1)_{0.16}(PEI)_{0.04}]ₙ₋

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃,
b, —NHCH₂CH₂OCOCH₂CH₂CONH-PEI),

δ 2.5˜2.7 (b, —NHCH₂CH₂OCOCH₂CH₂COOH,
b, —NHCH₂CH₂OCOCH₂CH₂CONH-PEI),

δ 2.9˜3.2 (b, —NHCH₂CH₂OCOCH₂CH₂COOH),

δ 3.4 (s, —NH(CH₂CH₂O)₁₆CH₃),

δ 3.4˜3.8 (b, —NH(CH₂CH₂O)₁₆CH₃),
b, —NHCH₂CH₂OCOCH₂CH₂CONH-PEI),

δ 3.9˜4.3 (b, —NHCH(CH(CH₃)CH₂CH₃)COOCH₂CH₃,
—NHCH₂CH₂OCOCH₂CH₂COOH)

Average molecular weight (M_{w}): 14000

### Example 10

### [NP(I)_{1.41}(P)_{0.40}(H1A1)_{0.11}(Imi)_{0.07}]ₙ

Poly(dichlorophosphazene) (10.00 g, 86.29 mmol), isoleucine ethyl ester (23.81 g, 121.67 mmol), ethanolamine (1.56 g, 25.89 mmol), aminomethoxy polyethylene glycol having the molecular weight of 750 (37.54 g, 50.05 mmol), triethylamine (90.98 ml, 652.77 mmol) and tetrahydrofuran (700 ml) were used according to the same procedure as Example 1 to give the final product [NP(I)_{1.45}(P)_{0.41}(H1)_{0.14}]ₙ (28 g, Yield 90%). Subsequently, [NP(I)_{1.45}(P)_{0.41}(H1)_{0.14}]ₙ (27.12 g) thus obtained was dissolved in tetrahydrofuran (700 ml) and reacted with 2 eq. of succinic anhydride (2.76 g) and 2 eq. of dimethylaminopyridine (3.37 g) at room temperature for 8 h. The reaction filtrate was concentrated under reduced pressure, dissolved in a small amount of methyl alcohol, dialyzed with methyl alcohol at room temperature for 5 days and with distilled water at 4° C. for 5 days, and dried at a low temperature to give the product [NP(I)_{1.32}(P)_{0.42}(H1A1)_{0.36}]ₙ (28 g, Yield 86%). Subsequently, [NP(I)_{1.32}(P)_{0.42}(H1A1)_{0.36}]ₙ (10 g) thus obtained was dissolved in tetrahydrofuran (400 ml). 10 eq. of diisopropylcarbodiimide (2.92 g) and 10 eq. of n-hydroxysuccinimide (2.67 g) each were dissolved in tetrahydrofuran (50 ml) and added to the polymer solution to activate the polymer for 40 min. Then, the tetrahydrofuran solution wherein 5 eq. of 1-(3-aminopropylimidazole) (1.45 g) was dissolved was added thereto. The resulting mixture was reacted in an ice bath for 5 h and then reacted at room temperature for 24 h.

Hydrogen Nuclear Magnetic Resonance Spectrum (CDCl₃, ppm):

δ 0.7˜1.1 (b, —NHCH(CH(CH₃)CH₂CH₃)COOCH₂CH₃),

δ 1.1˜1.3 (b, —NHCH(CH(CH₃)CH₂CH₃)COOCH₂CH₃),

δ 1.4˜1.8 (b, —NHCH(CH(CH₃)CH₂CH₃)COOCH₂CH₃),

δ 2.5˜2.7 (b, —NHCH₂CH₂OCOCH₂CH₂COOH,
b, —NHCH₂CH₂OCOCH₂CH₂CONH-Imi),

δ 2.9˜3.2 (b, —NHCH₂CH₂OCOCH₂CH₂COOH),

δ 3.4 (s, —NH(CH₂CH₂O)₁₆CH₃),

δ 3.4˜3.8 (b, —NH(CH₂CH₂O)₁₆CH₃),
b, —NHCH₂CH₂OCOCH₂CH₂CONH-Imi),

δ3.9˜4.3 (b, —NHCH(CH(CH₃)CH₂CH₃)COOCH₂CH₃,
b, —NHCH₂CH₂OCOCH₂CH₂COOH),

δ 6.8˜7.8 (b, —NHCH₂CH₂OCOCH₂CH₂CONH-Imi)

Average molecular weight (M_{w}): 14200

### Example 11

### Observation of the Temperature-Dependent Sol-Gel Phase Transition of the Phosphazene-Based Polymer

The phosphazene-based polymers prepared in Examples 1 to 9 were respectively dissolved in phosphate buffered saline (pH 7.4) at 4° C. to make the solutions with concentrations of 10 wt %. The solutions were put into a chamber of a Brookfield DV-III+ Rheometer equipped with an automatic thermostatic bath (TC-501). The temperature-dependent sol-gel phase transition was observed with raising the temperature at the rate of 0.04° C./min under the shear rate of 0.1 to 1.7 per second.

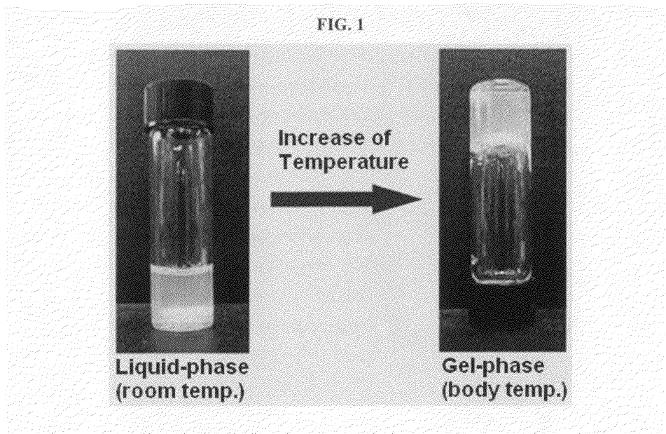

FIG. 1 is a photograph showing the temperature-dependent sol-gel phase transition of the phosphazene-based polymer according to the present invention. It shows that the polymer solution is in the fluid sol-phase at room temperature, but changes into the gel-phase at the body temperature.

The following Table 4 shows the test results of temperature-dependent gel characteristics of the thermosensitive phosphazene-based polymers of the present invention.

TABLE 4
Test results of temperature-dependent gel characteristics of the phosphazene-based
polymers

| | | Maximum gelling | Maximum gel |
|---|---|---|---|
| | | temperature. | solidity |
| Polymer | Structure | (° C.) | (Pa · s) |
| Example 1 | [NP(I)_{1.22}(P)_{0.57}(H1)_{0.21}]ₙ | 41 | 1015 |
| Example 2 | [NP(I)_{1.18}(P)_{0.42}(H2)_{0.40}]ₙ | 38 | 250 |
| Example 3 | [NP(I)_{1.36}(P)_{0.41}(H3)_{0.24}]ₙ | — | — |
| Example 4 | [NP(I)_{1.32}(P)_{0.42}(H1A1)_{0.36}]ₙ | 50 | 706 |
| Example 5 | [NP(I)_{1.32}(P)_{0.42}(H2A1)_{0.36}]ₙ | 37 | 153 |
| Example 6 | [NP(I)_{1.48}(P)_{0.41}(H1A2)_{0.11}]ₙ | 48 | 1213 |
| Example 7 | [NP(I)_{1.48}(P)_{0.33}(H1A2)_{0.19}]ₙ | 50 | 569 |
| Example 8 | [NP(I)_{1.53}(P)_{0.42}(H1A3)_{0.05}]ₙ | 36 | 406 |
| Example 9 | [NP(I)_{1.43}(P)_{0.37}(H1A1)_{0.16}(PEI)_{0.04}]ₙ | 60 | 1113 |
| Example 10 | [NP(I)_{1.41}(P)_{0.40}(H1A1)_{0.11}(Imi)_{0.07}]ₙ | 47 | 1450 |

In Table 4, the term ‘maximum gelling temperature’ refers to the temperature where the viscosity of the polymer solution reaches the maximum point, and the term ‘maximum gel solidity’ means the maximum viscosity of the polymer solution.

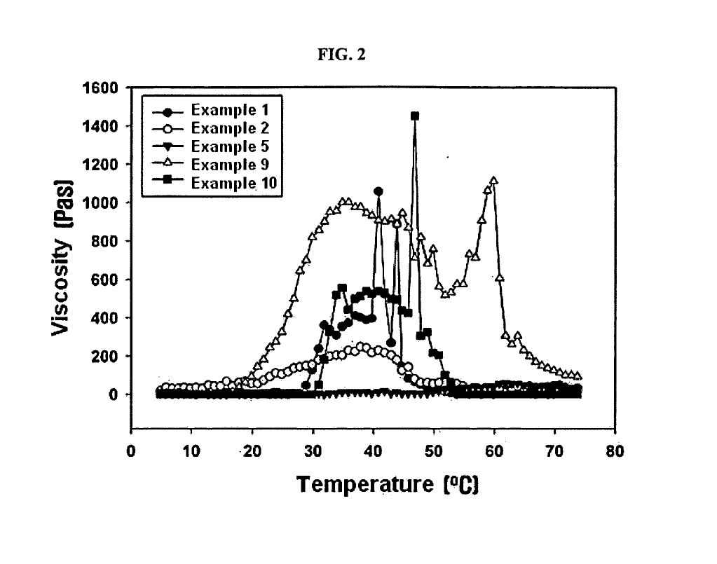

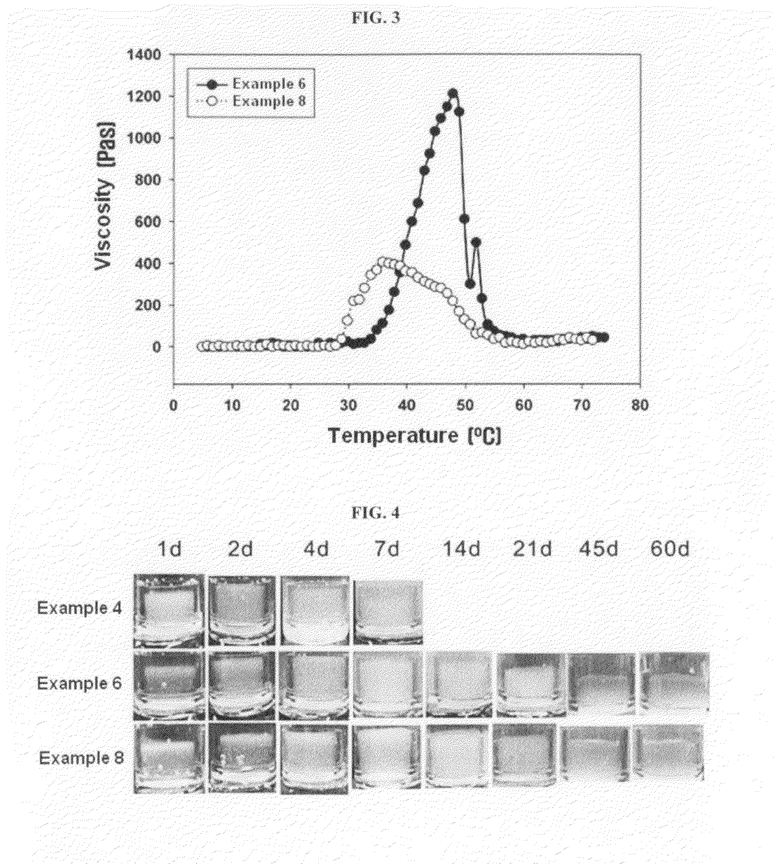

The temperature-dependent viscosity changes of the polyphosphazene-based polymers of the present invention are shown in FIGS. 2 and 3.

As is confirmed from Table 4 and FIG. 2, the phosphazene-based polymers show a broad spectrum of maximum gelling temperature and maximum gel solidity through the control of kinds of substituents having the ionic group by which the phosphazene-based polymer is substituted and the control of their compositions. In particular, the polymers of Examples 6 and 8 shown in FIG. 3 are prepared by reacting the hydroxy-containing phosphazene polymer obtained via the main synthetic reaction with glutaric anhydride and adipic anhydride, respectively. In the case of Example 8 wherein the more hydrophobic adipic anhydride is used, it has been confirmed to have the maximum gelling temperature that is lower than Example 6. Through this experiment, it has been demonstrated that the property of the polymer hydrogel changes depending on the kind of substituent.

### Example 12

### Observation of the Temperature-Dependent Decrease of Gel Amount of the Phosphazene-Based Polymers Substituted by Ionic Groups Different from Each Other

The phosphazene-based polymers prepared in the Examples of the present invention were dissolved in phosphate buffered saline (pH 7.4) to make the solutions with concentrations of 10 wt %, respectively, and allowed to stand in a water bath of 37° C. The decrease of gel amount with lapse of time was observed by the naked eye, and the remaining gel weighed to assess the decrease rate.

The time-dependent decrease of gel amount of the phosphazene-based polymers according to the present invention is represented in FIG. 3.

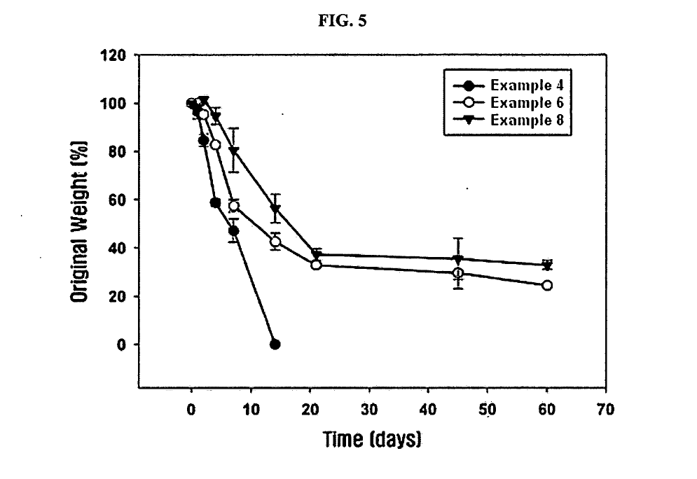

As can be seen from FIGS. 4 and 5, the phosphazene-based polymer having no carboxyl group according to Example 2 shows the molecular weight decrease of about 5% for the first 5 days and then the molecular weight decrease of 30% for 30 days. Examples 4, 6 and 7 all concern the polymers having a carboxyl group which is connected to an ester linker and they are different from each other in the carbon number present between the carboxyl group and the ester linker. The carbon number is 2 in Example 4, 3 in Example 6 and 4 in Example 7. In the case of phosphazene-based polymers having the mechanism that a gel phase is formed through the hydrophobic interaction, since the hydrophobic interaction may be facilitated by the carbon number change, it is understood that the carbon number exerts a great influence on the gel stability or degradation rate. As expected, in the case of Example 4 having the least carbon number, the amount of gel decreased to a half after 7 days and little gel remained after 14 days. In the case of Examples 6 and 8, the gel remained until about 60 days, and it was confirmed that the gel of Example 8 having the higher carbon number remained more as much as about 10%. From these results, it can be understood that the degradation rate and stability of the carboxy group-containing phosphazene-based polymer can be easily controlled by changing the degree of hydrophobicity of the substituent, in the addition of various substituents that can introduce a carboxyl group to the same phosphazene-based polymer, [NP(I)(P)(H1)].

### Example 13

### Observation of In Vivo Release Behavior of Human Growth Hormone in the Phosphazene-Based Polymer Hydrogel

The anionic human growth hormone and the cationic protamine sulfate were mixed in various ratios (protamine:human growth hormone=1:1 or 3:1) to prepare complexes. The phosphazene-based polymers of Examples 7 and 8 each were dissolved in phosphate buffered saline (pH 7.4) to make a solution with the concentration of 10 wt %, to which was added the above prepared complex. For the in vivo release test, 200 μg/kg of hydrogel containing human growth hormone in the concentration of 1.1 mg/kg was injected to test animal rats (OrientalBio, SD rat, male of 4-week old). At the predetermined time, 0.3 nm of blood was taken from the tail of rat and centrifuged. The amount of human growth hormone contained in the supernatant was measured by using an ELISA kit (BioCheck, Inc., USA).

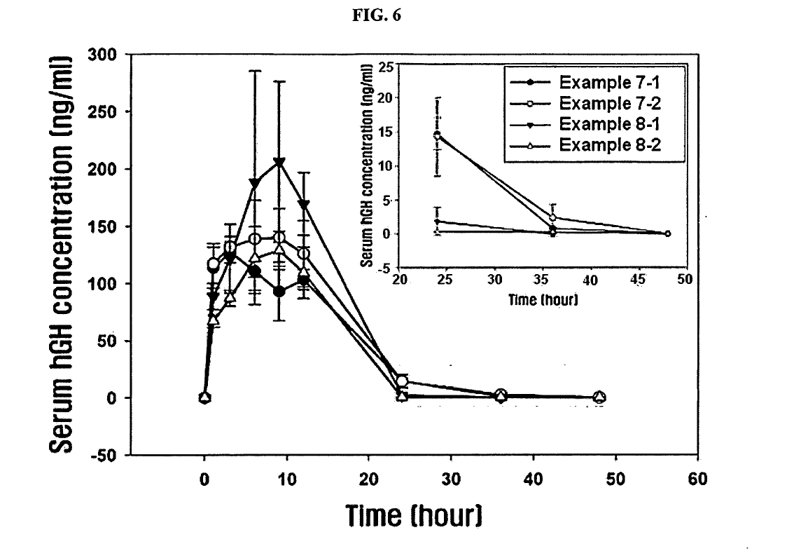

The ratio of protamine sulfate and human growth hormone in the formation of the complex was fixed to protamine:human growth hormone=1:1 or 3:1, and was represented in the format such as Example-1 (1:1) or Example-2 (3:1). The release behavior of human growth hormone in the phosphazene-based polymer hydrogel to which protamine was combined in various ratios was measured as above and is represented in FIG. 6. Although FIG. 6 does not show, when the solution containing human growth hormone only was injected, sharp decrease of blood concentration was observed within 24 h. On the contrary, when the hormone was combined with the phosphazene-based polymer hydrogel, sustained release as shown in the figure could be observed. Furthermore, when Examples 7 and 8 are compared, Example 8 which has one more carbon atom and thus shows more excellent stability and slower degradation rate in the body than Example 7 was identified to release human growth hormone for the time longer by about 20 h. From these results, it can be seen that even the release rate of the drug to be delivered can be controlled by the adjustment of degradation rate of the phosphazene-based polymer, which is achieved by the selection of substituent. Moreover, the release time required for a drug may be easily controlled by the substituent. Thus, the present invention is expected to be applicable in the delivery of various drugs.

As stated above, the polyphosphazene-based polymer hydrogel according to the present invention has the thermosensitivity of showing the temperature-dependent sol-gel phase transition, forms a gel phase at the body temperature when it is introduced into the body to make it easy to control the release of bioactive substances such as drugs, and has the functional groups capable of making chemical bonds such as ionic bond, covalent bond, coordinate bond, etc. with drugs and thus is excellent in bearing the drugs, etc. Furthermore, since it can control the degradation rate depending on the kind of substituent, it can selectively control the release time depending on the characteristics of drugs. Thus, it is very useful as a material for delivery of bioactive substances such as drugs, etc. and also is expected to be applicable in various industrial fields.

Also, the biodegradable and thermosensitive phosphazene-based polymer hydrogel as the drug delivery system according to the present invention not only increases the solubility of drug but also shows the release behavior which is different depending on the substituent during the in vivo drug release test. This is due to the differences in the degradation rate and stability caused by the kind of substituent which endows the phosphazene-based polymer with ionic property.

In view of the above, the drug or cell-containing biodegradable and thermosensitive phosphazene-based polymer according to the present invention can be easily administered into the body and its degradation rate can be easily controlled through the selection of a functional group. Thereby it can control the in vivo and ex vivo delivery rate of drugs or cells to obtain excellent effect in the therapies which are fit for each condition.

What is claimed is:

1. A phosphazene-based polymer represented by following Formula (1), or a pharmaceutically acceptable salt thereof: 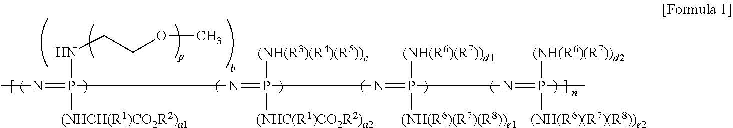
in which
p ranges from 16 to 50,
R¹ is selected from the group consisting of H, CH₃, CH₂SH, CH(CH₃)₂, CH₂CH(CH₃)₂, CH(CH₃)C₂H₅, CH₂CH₂SCH₃, CH₂C₆H₅, CH₂C₆H₄OH, and CH₂C₂NH₂C₆H₄,
R² is selected from the group consisting of CH₃, C₂H₅, C₃H₇, C₄H₉, CH₂C₆H₅, and CH₂CHCH₂,
R³ is CH(W),
R⁴ is selected from the group consisting of CO₂, CO₂CH₂CO₂, CO₂CH(CH₃)CO₂, and CONHCH(X)CO₂,
R⁵ is selected from the group consisting of H, CH₃, and C₂H₅,
wherein W and X are independently selected from the group consisting of H, HCH₂, CH₃, CH(CH₃)₂, CH₂CH(CH₃)₂, CH(CH₃)C₂H₅, CH₂CH₂SCH₃, CH₂C₆H₅, CH₂C₂NH₂C₆H₄, CO₂C₂H₅, (CH₂)₂CO₂C₂H₅, CH₂OH, CH(CH₃)OH, CH₂C₆H₄OH, CH₂COOH, CH₂CH₂COOH, CH₂CONH₂, C₄H₈NH₂, C₃H₆NHC(═NH)NH₂, CH₂C₃N₂H₃, and CH₂SH,
R⁶ is a divalent functional group derived from a hydroxyalkyl or hydroxy-containing amino acid, in which one hydrogen from the alkyl group or NH group from the amino acid is removed and one hydrogen is removed from the hydroxy group,
R⁷ is a monovalent functional group produced by the removal of one OH group from dicarboxylic acid-based compounds having 3 to 30 carbon atoms, or a divalent functional group produced by the removal of two OH groups from dicarboxylic acid-based compounds having 3 to 30 carbon atoms,
R⁸ is selected from the group consisting of a protecting group, NH₂CH(SH)CO₂H, NH₂(CH₂)_{q}SH, NH₂(CH₂CH₂NH)ᵣH, [NH₂CH(C₄H₈NH₂)CO]ᵣOH, [NH₂CH[(CH₂)₃C(═NH)(NH₂)]CO]ᵣOH, [OCH₂CH₂CH₂CH₂CH₂N(CH₂CH₂CO₂CH₂CH₂)₂]ᵣ, folic acid, hyaluronic acid, cyclodextrin, imidazole-based compound, anticancer agent, histidine, lysine, arginine, cysteine, thiolalkylamine, spermine, spermidine, polyethyleneimine, polyhistidine, polylysine, polyarginine, protamine, heparin, chitosan, and peptide consisting of 1 to 20 amino acids, wherein q ranges from 1 to 20, and r ranges from 1 to 18000,
a₁, a₂, b, c, d₁, d₂, e₁ and e₂ respectively represent the content of each substituent, wherein a₁, a₂, b, d₁ and d₂ respectively range from 0.01 to 1.9, c, e₁ and e₂ respectively range 0 to 1.9, and a₁+a₂+b+c+d₁+d₂+e₁+e₂=2.0,
n, which is the degree of polymerization of the polyphosphazene, ranges from 5 to 100000.

2. The phosphazene-based polymer or a pharmaceutically acceptable salt thereof according to claim 1, wherein the divalent functional group derived from a hydroxyalkyl or hydroxy-containing amino acid is selected from the group consisting of a divalent functional group derived from an alcohol having straight-chain or branched alkyl which has 1 to 30 carbon atoms and is unsubstituted or substituted by one or more substituents selected from the group consisting of halogen, C₁-C₁₂-alkoxy, acryloyloxy and amino acid; and a divalent functional group derived from an amino acid having hydroxy group

3. The phosphazene-based polymer or a pharmaceutically acceptable salt thereof according to claim 1, wherein the dicarboxylic acid-based compounds are selected from the group consisting of methylsuccinic acid, 3-3-dimethylglutaric acid, phenylsuccinic acid, aconitic acid, dimethylmaleic acid, itaconic acid, diglycolic acid, citraconic acid, glutaric acid, succinic acid, maleic acid, 2,2-dimethylsuccinic acid, 3-methylglutaric acid, phenylmaleic acid, 2-phenylglutaric acid, dodecenylsuccinic acid, dimethylmaleic acid, N—Z-L-aspartic acid, thiodiglycolic acid, tetrafluorosuccinic acid, cis-aconitic acid, 1-cyclopenten-1,2-dicarboxylic acid, phthalic acid, 3,6-dichlorophthalic acid and adipic acid.

4. The phosphazene-based polymer or a pharmaceutically acceptable salt thereof according to claim 1, wherein the imidazole-based compound is selected from the group consisting of dacarbazine, 1-(3-aminopropyl)imidazole, methylhistamine dihydrochloride, 4-(1H-imidazol-1-yl)aniline, histamine, imiquimod, biotin ethylenediamine, 2-(2-methylimidazolyl)ethylamine dihydrochloride, 5-amino-4-imidazolecarboxamide hydrochloride, 5-aminoimidazole-4-carboxamide, 4-imidazoleacrylic acid, 4-imidazolecarboxylic acid, 2-iminobiotin, L-(+)-ergothioneine, 4,5-imidazoledicarboxylic acid, 1-(2-hydroxyethyl)imidazole, 4(5)-(hydroxymethyl)imidazole, 4-imidazolemethanol hydrochloride, etanidazole, 4-(imidazol-1-yl)phenol, HMMNI (2-hydroxymethyl-1-methyl-5-nitro-1H-imidazole), 2-mercaptoimidazole, 1-(4-hydroxybenzyl)imidazole-2-thiol, thiabendazole, 1,1′-thiocarbonyldiimidazole, 2-mercapto-1-methylimidazole, methimazole, 1-(2,3,5,6-tetrafluorophenyl)imidazole, 1-(heptafluorobutyryl)imidazole, 1-(pentafluoropropionyl)imidazole, 1-(trifluoroacetyl)imidazole, 1-(trifluoromethanesulfonyl)imidazole, 1-[2-(trifluoromethyl)phenyl]imidazole, 2-bromo-1H-imidazole, 2-butyl-4-chloro-5-(hydroxymethyl)imidazole, 2-butyl-5-chloro-1H-imidazole-4-carboxaldehyde, 2-chloro-1H-imidazole, 4-(4-bromophenyl)-1H-imidazole, 4-(4-chlorophenyl)-1H-imidazole, 4-(4-fluorophenyl)-1H-imidazole, 5-bromo-1-methyl-1H-imidazole, 6-bromo-1H-benzimidazole, cyazofamid, imazalil, ketoconazole, fenobam, imazalil sulfate, losartan potassium, neurodazine, nutlin-3, SB 220025 trihydrochloride, SB 202190 (4-(4-fluorophenyl)-2-(4-hydroxyphenyl)-5-(4-pyridyl)-1H-imidazole), PD 169316 (4-(4-fluorophenyl)-2-(4-nitrophenyl)-5-(4-pyridyl)-1H-imidazole), SB 239063 (trans-1-(4-hydroxycyclohexyl)-4-(4-fluorophenyl)-5-(2-methoxypyridimidin-4-yl)imidazole), tioconazole, triflumizole, 2,4,5-tribromoimidazole, 5-chloro-1-methyl-4-nitroimidazole, 2-ethyl-4-methyl-1H-imidazole-1-propanenitrile, 4,5-dicyanoimidazole and 5-ethynyl-1-methyl-1H-imidazole.

5. The phosphazene-based polymer or a pharmaceutically acceptable salt thereof according to claim 1, wherein
R¹ is CH(CH₃)C₂H₅,
R² is C₂H₅,
R⁶ is —(CH₂)ₗO— (1 is an integer of 2 to 5),
R⁷ is —CO(CH₂)ₘCOO— or —CO(CH₂)ₘCOOH (m is an integer of 2 to 4),
R⁸ is polyethyleneimine, protamine or imidazole.

6. A phosphazene-based polymer hydrogel, which comprises a polymer solution wherein the phosphazene-based polymer or a pharmaceutically acceptable salt thereof according to claim 1 is dissolved in a solvent in the concentration of 1 to 50 wt %.

7. The phosphazene-based polymer hydrogel according to claim 6, wherein the solvent is one or more selected from the group consisting of water, buffer, acidic solution, basic solution, salt solution, physiological saline, water for injection, and dextrose saline.

8. The phosphazene-based polymer hydrogel according to claim 6, which shows the sol-gel phase transition at the temperature ranging from 5 to 70° C. and has the gel phase at the body temperature range.

9. A composition for bioactive substance delivery, which comprises the phosphazene-based polymer or a pharmaceutically acceptable salt thereof according to claim 1, or the phosphazene-based polymer hydrogel comprising a polymer solution wherein said phosphazene-based polymer or a pharmaceutically acceptable salt thereof is dissolved in a solvent in the concentration of 1 to 50 wt %.

10. The composition for bioactive substance delivery according to claim 9, which further comprises one or more additives selected from the group consisting of cationic polymer having the weight average molecular weight of 200 to 750,000, anionic polymer having the weight average molecular weight of 200 to 750,000, amino acid, peptide, protein, fatty acid, phospholipid, vitamin, drug, polyethylene glycol ester, steroid, amine compound, acrylic copolymer, organic solvent, preservative, sugar, polyol, sugar-containing polyol, sugar-containing amino acid, surfactant, sugar-containing ion, silicate, metal salt and ammonium salt in the amount of 1×10⁻⁶ to 30 wt %.

11. A bioactive substance delivery system, which comprises
the composition for bioactive substance delivery according to claim 9, and
one or more bioactive substances selected from the group consisting of a therapeutic cell, a protein, a polypeptide, a peptide, a vaccine, a gene, a hormone, an anticancer agent, and an angiogenesis inhibitor.

12. The bioactive substance delivery system according to claim 11, wherein said therapeutic cell is one or more selected from the group consisting of preosteoblast, chondrocyte, umbilical vein endothelial cell (UVEC), osteoblast, adult stem cell, schwann cell, oligodendrocyte, hepatocyte, mural cell (used in combination with UVEC), myoblast, insulin secreting cell, endothelial cell, smooth muscle cell, fibroblast, β cell, endodermal cell, hepatic stem cell, juxraglomerular cell, skeletal muscle cell, keratinocyte, melanocyte, langerhans cell, merkel cell, dermal fibroblast, and preadipocyte.

13. The bioactive substance delivery system according to claim 11, wherein
said protein, polypeptide and peptide are one or more selected from the group consisting of exendin-4, erythropoietin, interferon-alpha, interferon-beta, interferon-gamma, growth hormone, growth hormone releasing factor, nerve growth factor, G-CSF (granulocyte-colony stimulating factor), GM-CSF (granulocyte macrophage-colony stimulating factor), M-CSF (macrophage-colony stimulating factor), blood clotting factor, insulin, oxytocin, basopressin, adrenocorticotropic hormone, fibroblast growth factor, epidermal growth factor, platelet-derived growth factor, insulin-like growth factor, vascular endothelial growth factor, transforming growth factor, brain-derived neurotrophic factor, neurotrophin-3 (NT-3), neurotrophin-4/5, prolactin, luliberin, luteinizing hormone releasing hormone (LHRH), LHRH agonists, LHRH antagonists, somatostatin, glucagon, interleukin-2 (IL-2), interleukin-11 (IL-11), gastrin, tetragastrin, pentagastrin, urogastrone, secretin, calcitonin, enkephalins, endorphins, angiotensins, thyrotropin releasing hormone, tumor necrosis factor, tumor necrosis factor related apoptosis inducing ligand, heparinase, bone morphogenic protein, hANP (human atrial natriuretic peptide), glucagon-like peptide, renin, bradykinin, bacitracins, polymyxins, colistins, tyrocidine, gramicidins, cyclosporins, neurotensin, tachykinin, neuropeptide Y, peptide YY, vasoactive intestinal polypeptide, pituitray adenylate cyclase-activating polypeptide, antibodies against the above substances, enzymes, and cytokines,
said vaccine is a hepatitis vaccine,
said gene is one or more selected from the group consisting of small interfernce RNA (siRNA), plasmid DNA, and antisense oligodeoxynucleotide (AS-ODN),
said hormone is one or more selected from the group consisting of testosterone, estradiol, progesterone, and prostaglandins,
said anticancer agent is one or more selected from the group consisting of paclitaxel, doxorubicin, 5-fluorouracil, cisplatin, carboplatin, oxaliplatin, tegafur, irinotecan, docetaxel, cyclophosphamide, cemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansine, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassuim, medroxypogesterone acetate, epirubicin, letrozole, pirarubicin, topotecan, altretamine, toremifene citrate, BCNU, taxotere, actinomycin D, anasterozole, belotecan, imatinib, floxuridine, gemcitabine, hydroxyurea, zoledronate, vincristine, flutamide, valrubicin, streptozocin, and polyethylene glycol conjugated anticancer agents thereof, and
said angiogenesis inhibitor is one or more selected from the group consisting of clodronate, 6-deoxy-6-demethyl-4-dedimethylaminotetracycline (COL-3), doxycycline, marimastat, 2-methoxyestradiol, squalamine, thalidomide, TNP-470, combretastatin A4, soy isoflavone, enzastaurin, revimid, celecoxib, vandetanib, halofuginone hydrobromide, interferon-alpha, bevacizumab, shark cartilage extract, interleukin-12, vascular endothelial growth factor trap (VEFG-trap), cetuximab, rebimastat, matrix metalloproteinase (MMP) inhibitor, protein kinase C beta inhibitor, endostatin, vatalanib, sunitinib malate, cilenqitide, humanized monoclonal antibody, volociximab, and integrin alpha-5-beta-1 antagonists.

14. The bioactive substance delivery system according to claim 11, which further comprises one or more additives selected from the group consisting of cationic polymer having the weight average molecular weight of 200 to 750,000, anionic polymer having the weight average molecular weight of 200 to 750,000, amino acid, peptide, protein, fatty acid, phospholipid, vitamin, drug, polyethylene glycol ester, steroid, amine compound, acrylic copolymer, organic solvent, preservative, sugar, polyol, sugar-containing polyol, sugar-containing amino acid, surfactant, sugar-containing ion, silicate, metal salt and ammonium salt in the amount of 1×10⁻⁶ to 30 wt %.

15. A method for delivery of bioactive substances, comprising:
preparing the bioactive substance delivery system comprising the bioactive substance delivery composition according to claim 9 and one or more bioactive substances selected from the group consisting of a therapeutic cell, a protein, a polypeptide, a peptide, a vaccine, a gene, a hormone, an anticancer agent, and an angiogenesis inhibitor; and
administering the bioactive substance delivery system to a patient in need of the administration of the bioactive substance.

16. The method for delivery of bioactive substances according to claim 15, wherein said therapeutic cell is one or more selected from the group consisting of preosteoblast, chondrocyte, umbilical vein endothelial cell (UVEC), osteoblast, adult stem cell, schwann cell, oligodendrocyte, hepatocyte, mural cell (used in combination with UVEC), myoblast, insulin secreting cell, endothelial cell, smooth muscle cell, fibroblast, 13 cell, endodermal cell, hepatic stem cell, juxraglomerular cell, skeletal muscle cell, keratinocyte, melanocyte, langerhans cell, merkel cell, dermal fibroblast, and preadipocyte.

17. The method for delivery of bioactive substances according to claim 15, wherein
said protein, polypeptide and peptide are one or more selected from the group consisting of exendin-4, erythropoietin, interferon-alpha, interferon-beta, interferon-gamma, growth hormone, growth hormone releasing factor, nerve growth factor, G-CSF (granulocyte-colony stimulating factor), GM-CSF (granulocyte macrophage-colony stimulating factor), M-CSF (macrophage-colony stimulating factor), blood clotting factor, insulin, oxytocin, basopressin, adrenocorticotropic hormone, fibroblast growth factor, epidermal growth factor, platelet-derived growth factor, insulin-like growth factor, vascular endothelial growth factor, transforming growth factor, brain-derived neurotrophic factor, neurotrophin-3 (NT-3), neurotrophin-4/5, prolactin, luliberin, luteinizing hormone releasing hormone (LHRH), LHRH agonists, LHRH antagonists, somatostatin, glucagon, interleukin-2 (IL-2), interleukin-11 (IL-11), gastrin, tetragastrin, pentagastrin, urogastrone, secretin, calcitonin, enkephalins, endorphins, angiotensins, thyrotropin releasing hormone, tumor necrosis factor, tumor necrosis factor related apoptosis inducing ligand, heparinase, bone morphogenic protein, hANP (human atrial natriuretic peptide), glucagon-like peptide, renin, bradykinin, bacitracins, polymyxins, colistins, tyrocidine, gramicidins, cyclosporins, neurotensin, tachykinin, neuropeptide Y, peptide YY, vasoactive intestinal polypeptide, pituitray adenylate cyclase-activating polypeptide, antibodies against the above substances, enzymes, and cytokines,
said vaccine is a hepatitis vaccine,
said gene is one or more selected from the group consisting of small interfernce RNA (siRNA), plasmid DNA, and antisense oligodeoxynucleotide (AS-ODN),
said hormone is one or more selected from the group consisting of testosterone, estradiol, progesterone, and prostaglandins,
said anticancer agent is one or more selected from the group consisting of paclitaxel, doxorubicin, 5-fluorouracil, cisplatin, carboplatin, oxaliplatin, tegafur, irinotecan, docetaxel, cyclophosphamide, cemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansine, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassuim, medroxypogesterone acetate, epirubicin, letrozole, pirarubicin, topotecan, altretamine, toremifene citrate, BCNU, taxotere, actinomycin D, anasterozole, belotecan, imatinib, floxuridine, gemcitabine, hydroxyurea, zoledronate, vincristine, flutamide, valrubicin, streptozocin, and polyethylene glycol conjugated anticancer agents thereof, and
said angiogenesis inhibitor is one or more selected from the group consisting of clodronate, 6-deoxy-6-demethyl-4-dedimethylaminotetracycline (COL-3), doxycycline, marimastat, 2-methoxyestradiol, squalamine, thalidomide, TNP-470, combretastatin A4, soy isoflavone, enzastaurin, revimid, celecoxib, vandetanib, halofuginone hydrobromide, interferon-alpha, bevacizumab, shark cartilage extract, interleukin-12, vascular endothelial growth factor trap (VEFG-trap), cetuximab, rebimastat, matrix metalloproteinase (MMP) inhibitor, protein kinase C beta inhibitor, endostatin, vatalanib, sunitinib malate, cilenqitide, humanized monoclonal antibody, volociximab, and integrin alpha-5-beta-1 antagonists.

18. The method for delivery of bioactive substances according to claim 15, wherein the bioactive substance delivery system further comprises one or more additives selected from the group consisting of cationic polymer having the weight average molecular weight of 200 to 750,000, anionic polymer having the weight average molecular weight of 200 to 750,000, amino acid, peptide, protein, fatty acid, phospholipid, vitamin, drug, polyethylene glycol ester, steroid, amine compound, acrylic copolymer, organic solvent, preservative, sugar, polyol, sugar-containing polyol, sugar-containing amino acid, surfactant, sugar-containing ion, silicate, metal salt and ammonium salt in the amount of 1×10⁻⁶ to 30 wt %.
